(19) United States
(12) Patent Application Publication
(10) Pub. No.: US 2024/0260946 A1
(43) Pub. Date: Aug. 8, 2024

(54) System and Method for Controlling an Ultrasound Probe

(71) Applicant: Exo Imaging, Inc., Santa Clara, CA (US)

(72) Inventors: Roberto Ivan VEGA ROMERO, Edmonton (CA); Masood DEHGHAN, Edmonton (CA); Dornoosh Zonoobi, Edmonton (CA)

(21) Appl. No.: 18/165,803

(22) Filed: Feb. 7, 2023

Related U.S. Application Data
(63) Continuation of application No. PCT/US23/62043, filed on Feb. 6, 2023.

(51) Int. Cl. A61B 8/00

(52) U.S. Cl. CPC A61B 8/54; A61B 8/4245

(57) ABSTRACT

A computing device receives first imaging data acquired via an ultrasound probe during a first portion of a scan performed by the ultrasound probe. The first imaging data was acquired in accordance with a first set of imaging control parameters, which requires that a first subset of a plurality of transducers of the ultrasound probe are activated during the first portion of the scan. During a second portion of the scan performed by the ultrasound probe, in accordance with a determination that the first imaging data meets a first set of conditions associated with one or more quality requirements for the first, the computing device causes the ultrasound probe to acquire second imaging data in accordance with a second set of imaging control parameters. The second set of imaging control parameters requires that a second subset of the plurality of transducers are activated during the second portion of the scan.

### RELATED APPLICATIONS

This application is a continuation of PCT Patent Application No. PCT/US23/62043, filed Feb. 6, 2023, titled “System and Method for Controlling an Ultrasound Probe,” which is incorporated by reference herein in its entirety.

### TECHNICAL FIELD

The disclosed implementations relate generally to systems, methods, and devices for controlling an ultrasound probe.

### BACKGROUND

Ultrasound imaging is an imaging method that uses sound waves to produce images of structures within a patient's body. Because ultrasound images are captured in real-time, they can also show movement of the body's internal organs as well as blood flowing through the blood vessels. The images can provide valuable information for diagnosing and directing treatment for a variety of diseases and conditions.

### SUMMARY

Portable (e.g., handheld, and/or battery-operated) ultrasound devices are capable of producing high quality images because they contain many (e.g., hundreds or thousands) transducers that can each produce sound waves and receive the echoes for creating an ultrasound image. However, current portable ultrasound devices suffer from several drawbacks. For example, the device operating time per battery charge cycle is limited due to the power consumed by the transducers. Furthermore, the device can be prone to overheating due to high power consumption by the transducers. The high power consumption of the transducers during the ultrasound scan can also cause the probe itself to reach high temperatures that would pose discomfort or danger to the patient, and require significant cool down periods during the ultrasound scanning process. The overheating issues of the device and the probe sometimes also occur to non-handheld ultrasound stations, where transducers may operate at even higher powers and/or transducer densities than handheld or battery powered devices.

Presently, most ultrasound examinations are done by pressing a portion of an ultrasound device (e.g., an ultrasound probe or scanner) against a surface or inside a cavity of a patient's body, adjacent to the area being studied. In general, the process of acquiring a medical ultrasound image has two phases, namely the exploration phase and the acquisition phase. During the exploration phase, an operator moves the ultrasound probe around an area of a patient's body until the operator finds a location and pose of the probe that results in an image of the anatomical structures of interest with sufficiently high quality. The acquisition phase involves acquiring and saving additional frames in accordance with the location and pose of the probe identified at the end of the exploration phase, for future analysis. Identifying the location and pose of the probe suitable for the acquisition phase is challenging for the operation, and requires much experience and time on the part of the operator.

Accordingly, an improved ultrasound probe and corresponding operating methods are desirable. In particular, there is a need for an ultrasound probe with longer device operating time, and improved thermal management features to reduce device and probe overheating, reduce undesirable cool down time, improve patient safety and comfort, and/or prevent premature failure of components.

As disclosed herein, in some embodiments, a handheld ultrasound probe uses a first subset of transducers that is less than all of the available transducers during the exploration phase. The ultrasound probe uses a second subset of transducers (e.g., greater than the first subset, all of the transducers, etc.) during the acquisition phase. In other words, the ultrasound probe generates low-resolution images during the exploration phase, and then generates high-resolution images during the acquisition phase. Because it takes less power for the device to generate low-resolution images than high-resolution images, the disclosed device and method may reduce the power-related overheating and extends the battery life between charges, and at the same time, reduce cool down time and improve patient comfort and safety.

As disclosed herein, in some embodiments, the ultrasound probe (or a computing device communicatively connected to the ultrasound probe) automatically adjusts the power used in the ultrasound probe by changing the percentage of all the available transducers that need to be active at a given time, based on various criteria related to image quality requirements for a respective scan.

As disclosed herein, in some embodiments, the ultrasound device or the computing device coupled to the ultrasound probe provide guidance (e.g., during the exploration phase of the scan) to the operator on how to position the ultrasound probe so as to obtain a high-quality frame that contains the anatomical structures of interest.

Accordingly, in some embodiments, the disclosed device, system, and/or method advantageously improve user experience by increasing device operating time between battery charges, and reducing the amount of heat generated while the ultrasound probe is being used, thereby extending useful operating time before any probe cooldown is required. Operator and patient safety may also be improved.

The systems, methods, and devices of this disclosure have several innovative aspects, the desirable attributes disclosed herein may be derived from one or more of the innovative aspects individually or as a combination, in accordance with some embodiments.

In accordance with some embodiments, a method of controlling an ultrasound probe is performed at a computing device that includes one or more processors and memory. The method includes, during a first portion of a first scan performed by the ultrasound probe, receiving first imaging data acquired via the ultrasound probe. The first imaging data was acquired in accordance with a first set of imaging control parameters. The first set of imaging control parameters requires that a first subset of a plurality of transducers of the ultrasound probe are activated during the first portion of the first scan. The method includes, during a second portion, after the first portion, of the first scan performed by the ultrasound probe, in accordance with a determination that the first imaging data meets a first set of conditions associated with one or more quality requirements for the first, causing the ultrasound probe to acquire second imaging data in accordance with a second set of imaging control parameters. The second set of imaging control parameters requires that a second subset of the plurality of transducers, different from the first subset of the plurality of transducers, are activated during the second portion of the first scan following the first portion of the first scan.

In some embodiments, the method includes, in accordance with a determination that the first imaging data does not meet the first set of conditions associated with the one or more quality requirements for the first scan, and in accordance with a determination that the first imaging data meets a second set of conditions associated with the one or more quality requirements for the first scan, causing the ultrasound probe to acquire third imaging data in accordance with a third set of imaging control parameters during the second portion of the first scan following the first portion of the first scan, wherein the third set of imaging control parameters is different from the second set of imaging control parameters.

In some embodiments, the method includes, in accordance with a determination that the first imaging data does not meet the first set of conditions associated with the one or more quality requirements for the first scan, and in accordance with a determination that the first imaging data does not meet the second set of conditions associated with the one or more quality requirements for the first scan, causing the ultrasound probe to continue using the first set of imaging control parameters to acquire additional imaging data during the second portion of the first scan.

In some embodiments, the method includes, in accordance with a determination that the first imaging data does not meet the first set of conditions associated with the one or more quality requirements for the first scan: determining one or more operations for adjusting the ultrasound probe during the second portion of the first scan; and performing at least one of the one or more operations.

In some embodiments, performing at least one of the one or more operations include providing guidance for one or more recommended movements to be executed by the ultrasound probe.

In some embodiments, performing at least one of the one or more operations include adjusting one or more of the first set of imaging control parameters when acquiring a next ultrasound image using the ultrasound probe.

In some embodiments, the method includes, prior to receiving the first imaging data, determining a respective scan type for the first scan. The respective scan type corresponds to a respective target anatomical structure. The method includes selecting the first set of image control parameters based at least in part on the respective scan type that is determined for the first scan.

In some embodiments, the method includes determining the one or more quality requirements for the first scan according to the respective scan type for the first scan.

In some embodiments, determining whether the first imaging data meets the first set of conditions includes: determining a respective value of a first quality measure for a first ultrasound image in the first imaging data; and determining that the first imaging data meets the first set of conditions in accordance with a determination that the respective value of the first quality measure for the first ultrasound image exceeds a first threshold value for the first quality measure.

In some embodiments, determining whether the first imaging data meets the first set of conditions includes: predicting a respective value for a first quality measure for a next ultrasound image to be acquired in accordance with the first set of imaging control parameters; and determining that the first imaging data meets the first set of conditions in accordance with a determination that the respective value of the first quality measure for the next ultrasound image to be acquired in accordance with the first set of imaging control parameters would exceed a first threshold value for the first quality measure.

In some embodiments, the method includes predicting the respective value of the first quality measure for the next ultrasound image to be acquired based on a predicted trajectory of the ultrasound probe.

In accordance with some embodiments, a method of controlling an ultrasound probe is performed at a computing device that includes one or more processors and memory. The method includes, during a first portion of a first ultrasound scan, acquiring first imaging data via the ultrasound probe in accordance with a first set of imaging control parameters, including activating a first subset of a plurality of transducers of the ultrasound probe during the first portion of the first scan. The method includes, during a second portion, after the first portion, of the first scan performed by the ultrasound probe, in accordance with a determination that the first imaging data meets a first set of conditions associated with one or more quality requirements for the first scan, acquiring second imaging data via the ultrasound probe in accordance with a second set of imaging control parameters, including activating a second subset of the plurality of transducers, different from the first subset of the plurality of transducers, during the second portion of the first scan following the first portion of the first scan.

In some embodiments, the method includes, in accordance with a determination that the first imaging data does not meet the first set of conditions associated with the one or more quality requirements for the first scan, and in accordance with a determination that the first imaging data meets a second set of conditions associated with the one or more quality requirements for the first scan, acquiring third imaging data in accordance with a third set of imaging control parameters during the second portion of the first scan following the first portion of the first scan. The third set of imaging control parameters is different from the second set of imaging control parameters.

In some embodiments, the method includes, in accordance with a determination that the first imaging data does not meet the first set of conditions associated with the one or more quality requirements for the first scan, and in accordance with a determination that the first imaging data does not meet the second set of conditions associated with the one or more quality requirements for the first scan, continuing to use the first set of imaging control parameters to acquire additional imaging data during the second portion of the first scan.

In some embodiments, the method includes, prior to acquiring the first imaging data, determining a respective scan type for the first scan. The respective scan type corresponds to a respective target anatomical structure. The method includes selecting the first set of image control parameters based at least in part on the respective scan type that is determined for the first scan.

In some embodiments, determining whether the first imaging data meets the first set of conditions includes: determining a respective value of a first quality measure for a first ultrasound image in the first imaging data; and determining that the first imaging data meets the first set of conditions in accordance with a determination that the respective value of the first quality measure for the first ultrasound image exceeds a first threshold value for the first quality measure.

In some embodiments, determining whether the first imaging data meets the first set of conditions includes predicting a respective value for a first quality measure for a next ultrasound image to be acquired in accordance with the first set of imaging control parameters; and determining that the first imaging data meets the first set of conditions in accordance with a determination that the respective value of the first quality measure for the next ultrasound image to be acquired in accordance with the first set of imaging control parameters would exceed a first threshold value for the first quality measure

In accordance with some embodiments, a method of managing heat generation on an ultrasound probe is performed at a computing device that includes one or more processors and memory. The method includes, during a first portion of a first scan performed by the ultrasound probe, receiving first imaging data acquired via the ultrasound probe. The first imaging data was acquired in accordance with a first set of imaging control parameters. The first set of imaging control parameters requires that a first subset, less than all, of a plurality of transducers of the ultrasound probe are activated during the first portion of the first scan. The method includes, during a second portion, after the first portion, of the first scan performed by the ultrasound probe, in accordance with a determination that the first imaging data meets a first set of conditions associated with one or more quality requirements for the first scan, causing the ultrasound probe to acquire second imaging data in accordance with a second set of imaging control parameters. The second set of imaging control parameters requires that a second subset of the plurality of transducers are activated during a second portion of the first scan following the first portion of the first scan. The second subset of the plurality of transducers corresponds to a greater density of transducers among the plurality of transducers on the ultrasound probe than the first subset of transducers of the plurality of transducers.

In some embodiments, determining that the first imaging data meets the first set of conditions associated with one or more quality requirements for the first scan includes determining that the first imaging data includes imaging data acquired over a first period of time in which a respective quality of the imaging data for the one or more quality requirements increases from below a quality threshold to above the quality threshold and remains above the quality threshold.

In some embodiments, the method includes, in accordance with a determination that the first imaging data does not meet the first set of conditions associated with the one or more quality requirements for the first scan, and in accordance with a determination that the first imaging data meets a second set of conditions associated with the one or more quality requirements for the first scan, causing the ultrasound probe to acquire third imaging data in accordance with a third set of imaging control parameters during the second portion of the first scan following the first portion of the first scan, wherein the third set of imaging control parameters is different from the second set of imaging control parameters.

In some embodiments, the method includes, in accordance with a determination that the first imaging data does not meet the first set of conditions associated with the one or more quality requirements for the first scan, and in accordance with a determination that the first imaging data does not meet the second set of conditions associated with the one or more quality requirements for the first scan, causing the ultrasound probe to continue using the first set of imaging control parameters to acquire additional imaging data during the second portion of the first scan.

In accordance with some embodiments, an ultrasound probe comprises a plurality of transducers and a control unit. The control unit is configured to control the plurality of transducers during a scan performed with the ultrasound probe. The control unit is configured to: during a first portion of a first scan performed by the ultrasound probe, acquire first imaging data via the ultrasound probe in accordance with a first set of imaging control parameters, including activating a first subset of a plurality of transducers of the ultrasound probe during the first portion of the first scan. The control unit is configured to, during a second portion, after the first portion, of the first scan performed by the ultrasound probe, in accordance with a determination that the first imaging data meets a first set of conditions associated with one or more quality requirements for the first scan, acquire second imaging data via the ultrasound probe in accordance with a second set of imaging control parameters, including activating a second subset of the plurality of transducers, different from the first subset of the plurality of transducers, during the second portion of the first scan following the first portion of the first scan.

In accordance with some embodiments, an ultrasound probe comprises a plurality of transducers and a control unit. The control unit is configured to perform any of the methods disclosed herein.

In accordance with some embodiments, a computer system comprises one or more processors and memory. The memory stores memory storing instructions that, when executed by the one or more processors, cause the computer system to perform any of the methods disclosed herein.

In accordance with some embodiments of the present disclosure, a non-transitory computer readable storage medium stores computer-executable instructions. The computer-executable instructions, when executed by one or more processors of a computer system, cause the computer system to perform any of the methods disclosed herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an ultrasound system for imaging a patient, in accordance with some embodiments.

FIG. 2 illustrates a block diagram of an ultrasound device in accordance with some embodiments.

FIG. 3 illustrates a block diagram of a computing device in accordance with some embodiments.

FIG. 4 illustrates a workflow 400 for automatically adjusting the operating power of an ultrasound probe in accordance with some embodiments.

FIG. 5 illustrates a contrast in image quality between a full resolution or high resolution ultrasound image and a low resolution ultrasound image in accordance with some embodiments.

FIG. 6 illustrates an exemplary process for automatically assessing the quality of an ultrasound image and/or assigning an image quality score to an acquired image, in accordance with some embodiments.

FIG. 7 illustrates an exemplary process for providing guidance on positioning an ultrasound probe relative to a patient's body, in accordance with some embodiments.

FIG. 8 illustrates an exemplary process for providing guidance to obtain an ultrasound image that meets image quality requirements, in accordance with some embodiments.

FIGS. 9A-9C illustrate changes in projected images of a heart with changes in ultrasound probe positioning, in accordance with some embodiments.

FIG. 10 illustrates an exemplary process for providing user guidance to acquire a higher quality ultrasound image, in accordance with some embodiments.

FIG. 11 illustrates an input sequence of ultrasound images and their respective quality scores, in accordance with some embodiments.

FIGS. 12A-12E illustrate exemplary ultrasound images of a hip, in accordance with some embodiments.

FIGS. 13A-13D illustrate exemplary ultrasound images of a heart, in accordance with some embodiments.

FIGS. 14A-14C illustrates a flowchart diagram for a method of controlling an ultrasound probe, in accordance with some embodiments.

FIG. 15 illustrates a flowchart diagram for a method for controlling an ultrasound probe, in accordance with some embodiments.

FIG. 16 illustrates a flowchart diagram for a method of managing heat generation on an ultrasound probe in accordance with some embodiments.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring some of these specific details.

### DESCRIPTION OF IMPLEMENTATIONS

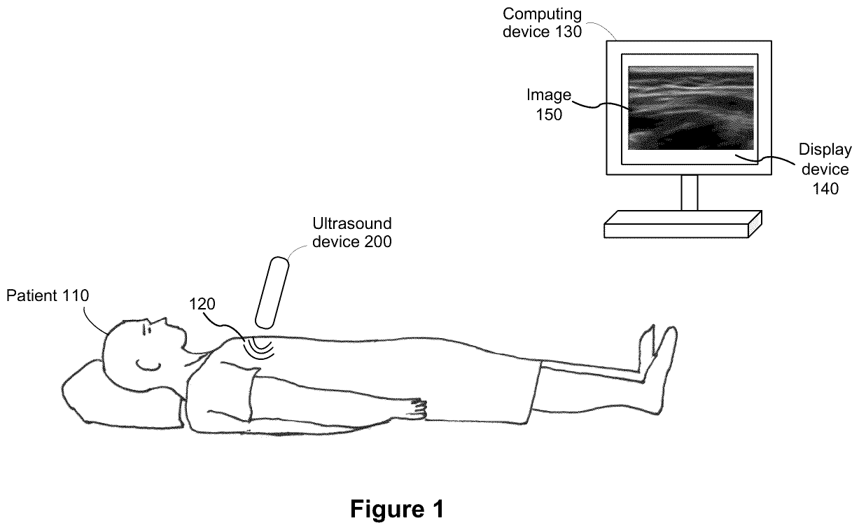

FIG. 1 illustrates an ultrasound system for imaging a patient, in accordance with some embodiments.

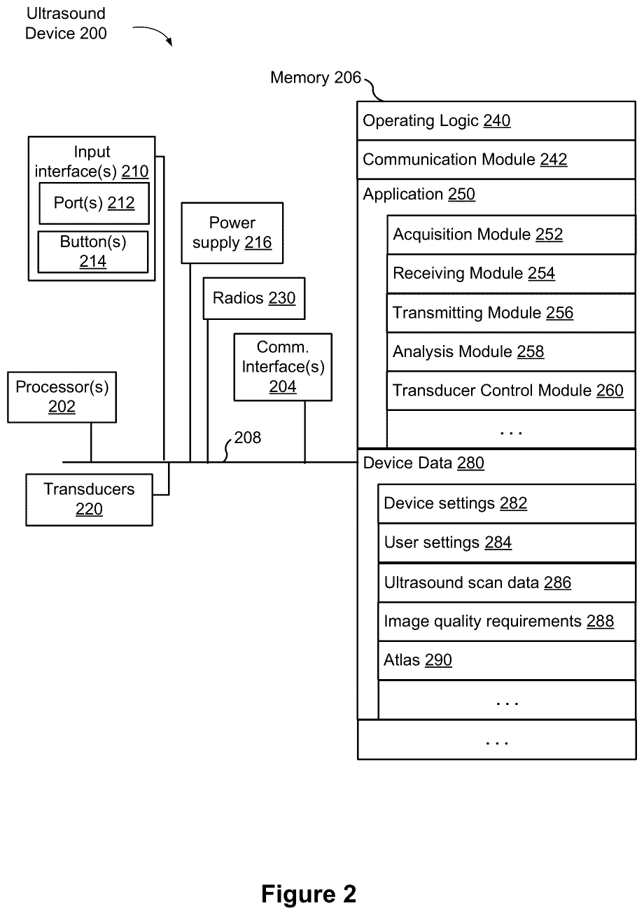

In some embodiments, a ultrasound device 200 is a portable, handheld device. In some embodiments, the ultrasound device 200 includes a probe portion that includes transducers (e.g., transducers 220, FIG. 2). In some embodiments, the transducers are arranged in an array. In some embodiments, the ultrasound device 200 includes an integrated control unit and user interface. In some embodiments, the ultrasound device 200 includes a probe that communicates with a control unit and user interface that is external to the housing of the probe itself. During operation, the ultrasound device 200 (e.g., via the transducers) produces sound waves 120 that are transmitted toward an organ, such as a heart or a lung, of a patient 110. The internal organ, or other object(s) to be imaged, may reflect a portion of the sound waves toward the probe portion of the ultrasound device 200, which are received by the transducers 220. In some embodiments, the ultrasound device 200 transmits the received signals to a computing device 130, which uses the received signals to create an image 150 that is also known as a sonogram. In some embodiments, the computing device 130 includes a display device 140 for displaying ultrasound images, and other input and output devices (e.g., keyboard, touch screen, joystick, touchpad, and/or speakers).

FIG. 2 illustrates a block diagram of an exemplary ultrasound device 200 in accordance with some embodiments.

In some embodiments, the ultrasound device 200 includes one or more processors 202, one or more communication interfaces 204 (e.g., network interface(s)), memory 206, and one or more communication buses 208 for interconnecting these components (sometimes called a chipset).

In some embodiments, the ultrasound device 200 includes one or more input interfaces 210 that facilitate user input. For example, in some embodiments, the input interfaces 210 include port(s) 212 and button(s) 214. In some embodiments, the port(s) can be used for receiving a cable for powering or charging the ultrasound device 200, or for facilitating communication between the ultrasound device and other devices (e.g., computing device 130, computing device 300, display device 140, printing device, and/or other input output devices and accessories).

In some embodiments, the ultrasound device 200 includes a power supply 216. For example, in some embodiments, the ultrasound device 200 is battery-powered. In some embodiments, the ultrasound device is powered by a continuous AC power supply.

In some embodiments, the ultrasound device 200 includes a probe portion that includes transducers 220, which may also be referred to as transceivers or imagers. Examples of transducers 220 include, without limitation, piezoelectric micromachined ultrasonic transducers (PMUT) and capacitive micromachined ultrasonic transducers (CMUT). In some embodiments, the transducers 220 are based on photo-acoustic or ultrasonic effects. For ultrasound imaging, the transducers 220 transmit ultrasonic waves towards a target (e.g., a target organ, blood vessels, etc.) to be imaged. The transducers 220 receive reflected sound waves (e.g., echoes) that bounce off body tissues. The reflected waves are then converted to electrical signals and/or ultrasound images. In some embodiments, the probe portion of the ultrasound device 200 is separately housed from the computing and control portion of the ultrasound device. In some embodiments, the probe portion of the ultrasound device 200 is integrated in the same housing as the computing and control portion of the ultrasound device 200. In some embodiments, part of the computing and control portion of the ultrasound device is integrated in the same housing as the probe portion, and part of the computing and control portion of the ultrasound device is implemented in a separate housing that is coupled communicatively with the part integrated with the probe portion of the ultrasound device. In some embodiments, the probe portion of the ultrasound device has a respective transducer array that is tailored to a respective scanner type (e.g., linear, convex, endocavitary, phased array, transesophageal, 3D, and/or 4D). In the present disclosure, “ultrasound probe” may refer to the probe portion of an ultrasound device, or an ultrasound device that includes a probe portion.

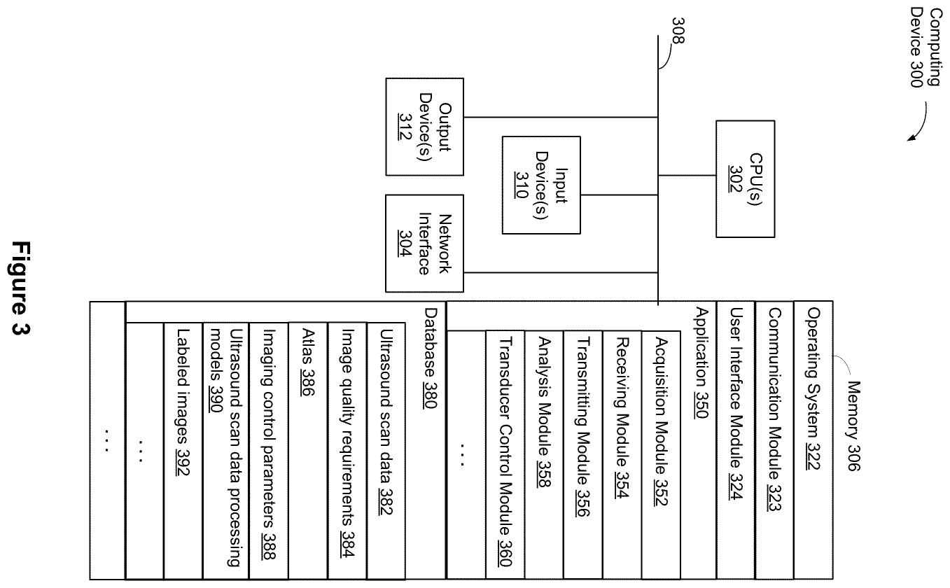

In some embodiments, the ultrasound device 200 includes radios 230. The radios 230 enable one or more communication networks, and allow the ultrasound device 200 to communicate with other devices, such as the computing device 130 in FIG. 1, the display device 140 in FIG. 1, and/or the computing device 300 in FIG. 3. In some implementations, the radios 230 are capable of data communications using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), software defined radio (SDR) etc.) custom or standard wired protocols (e.g., Ethernet, HomePlug, etc.), and/or any other suitable communication protocol, including communication protocols not yet developed as of the filing date of this document.

The memory 206 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 206, optionally, includes one or more storage devices remotely located from one or more processor(s) 202. The memory 206, or alternatively the non-volatile memory within the memory 206, includes a non-transitory computer-readable storage medium. In some implementations, the memory 206, or the non-transitory computer-readable storage medium of the memory 206, stores the following programs, modules, and data structures, or a subset or superset thereof:
operating logic 240 including procedures for handling various basic system services and for performing hardware dependent tasks;
a communication module 242 (e.g., a radio communication module) for connecting to and communicating with other network devices (e.g., a local network, such as a router that provides Internet connectivity, networked storage devices, network routing devices, server systems, computer device 130, computer device 300, and/or other connected devices etc.) coupled to one or more communication networks via the communication interface(s) 204 (e.g., wired or wireless);
application 250 for acquiring ultrasound data (e.g., imaging data) of a patient, and/or for controlling one or more components of the ultrasound device 200 and/or other connected devices (e.g., in accordance with a determination that the ultrasound data meets, or does not meet, certain conditions). In some embodiments, the application 250 includes:
   an acquisition module 252 for acquiring ultrasound data. In some embodiments, the ultrasound data includes imaging data. In some embodiments, the acquisition module 252 activates the transducers 220 (e.g., less than all of the transducers 220, different subset(s) of the transducers 220, all the transducers 220, etc.) according to whether the ultrasound data meets one or more conditions associated with one or more quality requirements;
   a receiving module 254 for receiving ultrasound data;
   a transmitting module 256 for transmitting ultrasound data to other device(s) (e.g., a server system, computer device 130, computer device 300, display device 140, and/or other connected devices etc.);
   an analysis module 258 for analyzing whether the data (e.g., imaging data) acquired by the ultrasound device 200 meets one or more conditions associated with quality requirements for an ultrasound scan. For example, in some embodiments, the one or more conditions include one or more of: a condition that the imaging data includes one or more newly acquired images that meet one or more threshold quality scores, a condition that the imaging data includes one or more newly acquired images that correspond to one or more anatomical planes that match a desired anatomical plane of a target anatomical structure, a condition that the imaging data includes one or more newly acquired images that include one or more landmark/features (or a combination of landmarks/features), a condition that the imaging data includes one or more newly acquired images that include a feature having a particular dimension, a condition that the imaging data supports a prediction that an image meeting one or more requirements would be acquired in the next one or more image frames, a condition that the imaging data supports a prediction that a first change (e.g., an increase by a percentage, or number) in the number of transducer used would support an improvement in the quality score of an image acquired in the next one or more image frames, and/or other analogous conditions; and
   a transducer control module 260 for activating (e.g., adjusting) a number of transducers 220 during portions of an ultrasound scan based on a determination that the ultrasound data meets (or does not meet) one or more quality requirements. For example, in some embodiments, the transducer control module 260 activates a first subset of the transducers 220 during the first portion of an ultrasound scan. In some embodiments, the transducer control module 260 activates a second subset of the transducers 220, different from the first subset of the transducers, during a second portion of the scan following the first portion of the scan, when the imaging data corresponding to the first portion of the scan meets (or does not meet) one or more quality requirements. In some embodiments, the transducer control module 260 controls one or more operating modes of the ultrasound device 200. For example, in some embodiments, the ultrasound device 200 is configured to operate in one or more low-power modes. In a respective low-power mode, the transducer control module 260 activates only a subset (e.g., 10%, 15%, 20%, or other preset subsets) of all the available transducers 220 in the ultrasound device 200. In some embodiments, the ultrasound device 200 is configured to operate in a full-power mode. In the full-power mode, the transducer control module 260 activates all the available transducers 220 to acquire a high-quality image; and
device data 280 for the ultrasound device 200, including but not limited to:
   device settings 282 for the ultrasound device 200, such as default options and preferred user settings. In some embodiments, the device settings 282 include imaging control parameters. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   user settings 284, such as a preferred gain, depth, zoom, and/or focus settings;
   ultrasound scan data 286 (e.g., imaging data) that are acquired (e.g., detected, measured) by the ultrasound device 200 (e.g., via transducers 220);
   image quality requirements data 288. In some embodiments, the image quality requirements data 288 include clinical requirements for determining the quality of an ultrasound image; and
   an atlas 290. In some embodiments, the atlas 290 includes anatomical structures of interest. In some embodiments, the atlas 290 includes three-dimensional representations of the anatomical structure of interest (e.g., hip, heart, lung, and/or other anatomical structures).

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above. Furthermore, the memory 206 may store additional modules or data structures not described above. In some embodiments, a subset of the programs, modules, and/or data stored in the memory 206 are stored on and/or executed by a server system, and/or by an external device (e.g., computing device 130 or computing device 300).

FIG. 3 illustrates a block diagram of a computing device 300 in accordance with some embodiments.

In some embodiments, the computing device 300 is a server or control console that is in communication with the ultrasound device 200 (e.g. ultrasound probe). In some embodiments, the computing device 300 is integrated into the same housing as the ultrasound device 200. In some embodiments, the computing device is a smartphone, tablet device, a gaming console, or other portable computing devices. In some embodiments, the computing device 300 may be provided by a combination of components integrated into the same housing as the ultrasound device 200, and a smartphone, tablet device, a gaming console, or other portable computing devices.

The computing device 300 includes one or more processors 302 (e.g., processing units of CPU(s)), one or more network interfaces 304, memory 306, and one or more communication buses 308 for interconnecting these components (sometimes called a chipset), in accordance with some implementations.

In some embodiments, the computing device 300 includes one or more input devices 310 that facilitate user input, such as a keyboard, a mouse, a voice-command input unit or microphone, a touch screen display, a touch-sensitive input pad, a gesture capturing camera, or other input buttons or controls. In some embodiments, the computing device 300 uses a microphone and voice recognition or a camera and gesture recognition to supplement or replace the keyboard. In some embodiments, the computing device 300 includes one or more output devices 312 that enable presentation of user interfaces and display content, such as one or more speakers and/or one or more visual displays (e.g., display device 140).

The memory 306 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 306, optionally, includes one or more storage devices remotely located from the one or more processors 302. The memory 306, or alternatively the non-volatile memory within the memory 306, includes a non-transitory computer-readable storage medium. In some implementations, the memory 306, or the non-transitory computer-readable storage medium of the memory 306, stores the following programs, modules, and data structures, or a subset or superset thereof:
an operating system 322 including procedures for handling various basic system services and for performing hardware dependent tasks;
a communication module 323 (e.g., a radio communication module) for connecting to and communicating with other network devices (e.g., a local network, such as a router that provides Internet connectivity, networked storage devices, network routing devices, server systems, computer device 130, ultrasound device 200, and/or other connected devices etc.) coupled to one or more communication networks via the network interface 304 (e.g., wired or wireless);
a user interface module 324 for enabling presentation of information (e.g., a graphical user interface for presenting application(s), widgets, websites and web pages thereof, games, audio and/or video content, text, etc.) either at the computing device 300 or another device;
application 350 for acquiring ultrasound data (e.g., imaging data) from a patient. In some embodiments, the application 350 is used for receiving data (e.g., ultrasound data, imaging data, etc.) acquired via an ultrasound device 200. In some embodiments, the application 350 is used for controlling one or more components of an ultrasound device 200 (e.g., the probe portion, and/or the transducers) and/or other connected devices (e.g., in accordance with a determination that the data meets, or does not meet, certain conditions). In some embodiments, the application 350 includes:
   an acquisition module 352 for acquiring ultrasound data. In some embodiments, the ultrasound data includes imaging data acquired by an ultrasound probe. In some embodiments, the acquisition module 352 activates the transducers 220 (e.g., less than all of the transducers 220, different subset(s) of the transducers 220, all the transducers 220, etc.) according to whether the ultrasound data meets one or more conditions associated with one or more quality requirements. In some embodiments, the acquisition module 352 causes the ultrasound device 200 to activate the transducers 220 (e.g., less than all of the transducers 220, different subset(s) of the transducers 220, all the transducers 220, etc.) according to whether the ultrasound data meets one or more conditions associated with one or more quality requirements.;
   a receiving module 354 for receiving ultrasound data. In some embodiments, the ultrasound data includes imaging data acquired by an ultrasound probe;
   a transmitting module 356 for transmitting ultrasound data (e.g., imaging data) to other device(s) (e.g., a server system, computer device 130, display device 140, ultrasound device 200, and/or other connected devices etc.);
   an analysis module 358 for analyzing whether the data (e.g., imaging data, power consumption data, and other data related to the acquisition process) (e.g., received by the ultrasound probe) meets one or more conditions associated with quality requirements for an ultrasound scan. For example, in some embodiments, the one or more conditions include one or more of: a condition that the imaging data includes one or more newly acquired images that meet one or more threshold quality scores, a condition that the imaging data includes one or more newly acquired images that correspond to one or more anatomical planes that match a desired anatomical plane of a target anatomical structure, a condition that the imaging data includes one or more newly acquired images that include one or more landmark/features (or a combination of landmarks/features), a condition that the imaging data includes one or more newly acquired images that include a feature having a particular dimension, a condition that the imaging data supports a prediction that an image meeting one or more requirements would be acquired in the next one or more image frames, a condition that the imaging data supports a prediction that a first change (e.g., an increase by a percentage, or number) in the number of transducer used would support an improvement in the quality score of an image acquired in the next one or more image frames, and/or other analogous conditions; and
   a transducer control module 360 for activating (e.g., adjusting, controlling, and/or otherwise modifying one or more operations of the transducers), or causing the ultrasound device 200 to activate (e.g., via the transducer control module 260), a number of transducers 220 during portions of an ultrasound scan based on a determination that the ultrasound data meets (or does not meet) one or more quality requirements. For example, in some embodiments, the transducer control module 360 activates a first subset of the transducers 220 during the first portion of an ultrasound scan. In some embodiments, the transducer control module 360 activates a second subset of the transducers 220, different from the first subset of the transducers, during a second portion of the scan following the first portion of the scan, when the imaging data corresponding to the first portion of the scan meets (or does not meet) one or more quality requirements. In some embodiments, the transducer control module 360 controls one or more operating modes of the ultrasound device 200. For example, in some embodiments, the ultrasound device 200 is configured to operate in a low-power mode. In the low-power mode, the transducer control module 360 activates only a subset (e.g., 10%, 15%, 20%, etc.) of all the available transducers 220 in the ultrasound device 200. In some embodiments, the ultrasound device 200 is configured to operate in a full-power mode. In the full-power mode, the transducer control module 360 activates all the available transducers 220 to acquire a high-quality image; and
a database 380, including:
   ultrasound scan data 382 (e.g., imaging data) that are acquired (e.g., detected, measured) by one or more ultrasound probes 200;
   image quality requirements data 384. In some embodiments, the image quality requirements data 384 include clinical requirements for determining the quality of an ultrasound image;
   an atlas 386. In some embodiments, the atlas 386 includes anatomical structures of interest. In some embodiments, the atlas 386 includes three-dimensional representations of the anatomical structure of interest (e.g., hip, heart, or lung);
   imaging control parameters 388. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   ultrasound scan data processing models 390 for processing ultrasound data. For example, in some embodiments, the ultrasound scan data processing models 390 are trained neural network models that are trained to determine whether an ultrasound image meets quality requirements corresponding to a scan type, or trained to output an anatomic plane corresponding to an anatomical structure of an ultrasound image, or trained to predict, based on a sequence of ultrasound images and their quality scores, whether a subsequent frame to be acquired by an ultrasound probe will contain certain anatomical structures and/or landmarks of interest; and
   labeled images 392 (e.g., a databank of images), including images for training the models that are used for processing new ultrasound data, and/or new images that have been or need to be processed. In some embodiments, the labeled images 392 are images of anatomical structures that have been labeled with their respective identifiers and relative positions.

Each of the above identified elements may be stored in one or more of the memory devices described herein, and corresponds to a set of instructions for performing the functions described above. The above identified modules or programs need not be implemented as separate software programs, procedures, modules or data structures, and thus various subsets of these modules may be combined or otherwise re-arranged in various embodiments. In some embodiments, the memory 306, optionally, stores a subset of the modules and data structures identified above. Furthermore, the memory 306 optionally stores additional modules and data structures not described above. In some embodiments, a subset of the programs, modules, and/or data stored in the memory 306 are stored on and/or executed by the ultrasound probe 200.

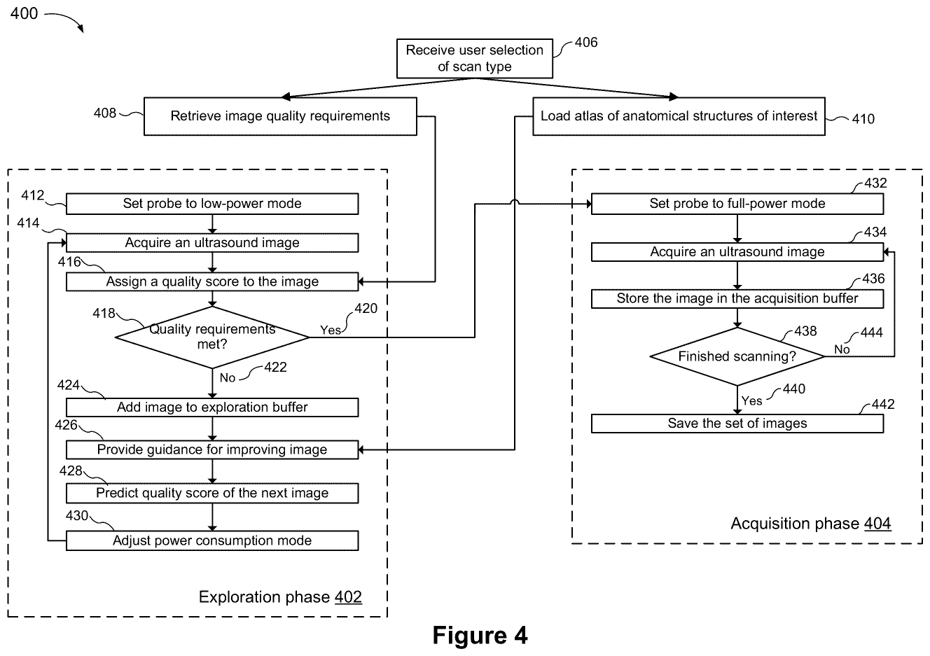

FIG. 4 illustrates a workflow 400 for automatically adjusting the operating power of an ultrasound probe, in accordance with some embodiments. In some embodiments, the workflow 400 is performed by one or more processors (e CPU(s) 302) of a computing device that is communicatively connected with an ultrasound probe. For example, in some embodiments, the computing device is a server or control console (e.g., a server, a standalone computer, a workstation, a smart phone, a tablet device, or a medical system) that is in communication with the ultrasound probe. In some embodiments, the computing device is a control unit integrated with the ultrasound probe in the same housing. In some embodiments, the ultrasound probe is a handheld ultrasound device, or a probe portion of an ultrasound scanning system.

FIG. 4 illustrates that, in some embodiments, the workflow 400 includes an exploration phase 402 and an acquisition phase 404. In the exploration phase 402, a user (e.g., operator) of the ultrasound device moves the probe around a surface of a patient until the user finds a good quality frame (e.g., an ultrasound image frame that meet one or more quality requirements and/or other conditions) that displays the anatomical structure(s) of interest. According to some embodiments of the present disclosure, a subset (e.g., less than all) (e.g., 10%, 25%, 30%, etc.) of the available transducers (e.g., transducers 220) of the ultrasound probe is activated during the exploration phase. According to some embodiments of the present disclosure, the ultrasound probe acquires one or more good quality frames (e.g., individual ultrasound image frames or a sequence of frames that meet one or more quality requirements and/or other conditions (optionally, different requirements and conditions used in the exploration phase)) in the acquisition phase, which is then saved (e.g., in device data 280 or database 380, etc.) for future analysis.

According to some embodiments of the present disclosure, the ultrasound probe acquires low-resolution images during the exploration phase 402, and acquires high-resolution images during the acquisition phase 404, e.g., due to the different numbers and densities of transducers used in the two phases. Since low-resolution images require less power than high-resolution images, the various embodiments described in the present disclosure address the technical problems of power-related overheating and short battery life of an ultrasound probe, by reducing the power-related overheating issues and extending the battery life of the ultrasound probe between charges. In addition, in some cases, the cool down period may be reduced, resulting in reduced total scan time.

Referring back to FIG. 4, in some embodiments, the workflow 400 includes receiving (406) user selection of a scan type. For example, in some embodiments, a user of the ultrasound probe initiates the scan process by determining the desired scan type. Nonlimiting examples of scan types include transversal view of the hip, a four-chamber echocardiogram, or a longitudinal scan of the lung. Different scan types correspond to different sets of operating parameters (e.g., frequency, phase, duration, movement direction, and/or transducer array type) of the ultrasound probe and/or requirements (e.g., presence, positions, sizes, and/or spatial relationships of landmarks selected based on scan type, clarity of image, and/or other hidden requirements based on machine learning or other image quality scoring methods) on the ultrasound images.

In some embodiments, the workflow 400 includes retrieving (408) a set of image quality requirements in accordance with user selection of the scan type. In some embodiments, the image quality requirements comprise clinical requirements for determining the quality of an image. In general, each scan type has its own clinical requirements. As a first nonlimiting example, the clinical requirements for an ultrasound image of a hip to determine the presence of hip dysplasia requires presence of the labrum, ischium, the midportion of the femoral head, flat and horizontal ilium, and absence of motion artifact. As another nonlimiting example, the clinical requirements for an echocardiography 4-chamber apical view are: (i) a view of the four chambers (left ventricle, right ventricle, left atrium, and right atrium) of the heart, (ii) the apex of the left ventricle is at the top and center of the sector, while the right ventricle is triangular in shape and smaller in area, (iii) myocardium and mitral leaflets should be visible, and (iv) the walls and septa of each chamber should be visible. Different sets of requirements for the operating parameters and/or image quality requirements may be implemented based on different scan types, in accordance with various embodiments.

In some embodiments, the workflow 400 includes loading (410) an atlas of anatomical structure(s) of interest. For example, in some embodiments, the atlas includes a three-dimensional representation of the anatomical structure of interest (e.g., hip, heart, or lung), corresponding to the selected scan type. The atlas is used to determine image quality and/or providing guidance to improve image quality during the exploration phase, in some embodiments.

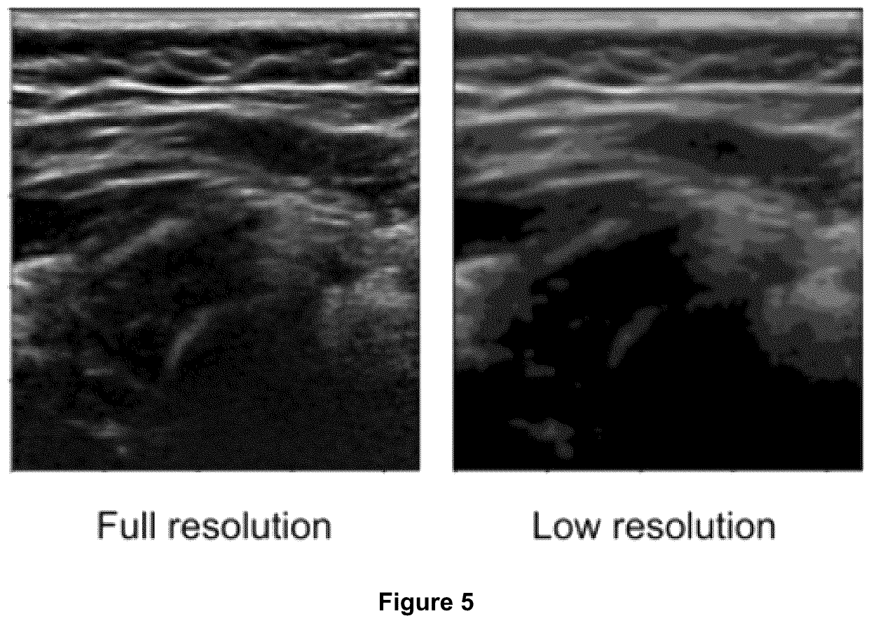

In some embodiments, the workflow 400 includes setting (412) the ultrasound device 200 (or causing the ultrasound device 200 to be set) to a low-power mode, and acquiring (414) an ultrasound image via the ultrasound probe while the probe is operating in the low-power mode. In some embodiments, the ultrasound probe activates a subset (i.e., less than all) of all the available transducers in the ultrasound probe (e.g., 10%, 15%, 20%, or 25%) while operating in the low-power mode. FIG. 5 illustrates, on the left, a full resolution ultrasound image that is generated using all available transducers of an ultrasound probe. FIG. 5 also illustrates, on the right, a low resolution ultrasound image that is generated using 10% of the available transducers of the same ultrasound probe. As shown in FIG. 5, the full resolution image provides more details and contrasts for details of an imaged part of the patient's anatomy, as compared to the low resolution image. However, the low resolution image nonetheless is useful in assessing (e.g., in the context of quality assessment based on machine learning techniques) whether the ultrasound probe is located at the correct location and/or has the correct pose for a desired scan type when the image is acquired. The quality score of the low resolution image is reflective of the likelihood that a good quality image frame or sequence of frames would be acquired using the full set of transducers if the present location and pose of the ultrasound probe is used (e.g., without further movement, or as a starting point of a full sweep).

In some embodiments, the workflow 400 includes assigning (418) a quality score to an ultrasound image that is acquired in step 414 (e.g., an image acquired in the low-power mode). In some embodiments, assigning the quality score to an ultrasound image occurs automatically in response to acquisition of the ultrasound image during the scan. In some embodiments, image quality is assessed one frame at a time. In some embodiments, image quality for a newly acquired image is assessed based on the newly acquired image, as well as a sequence of one or more images acquired right before the newly acquired image.

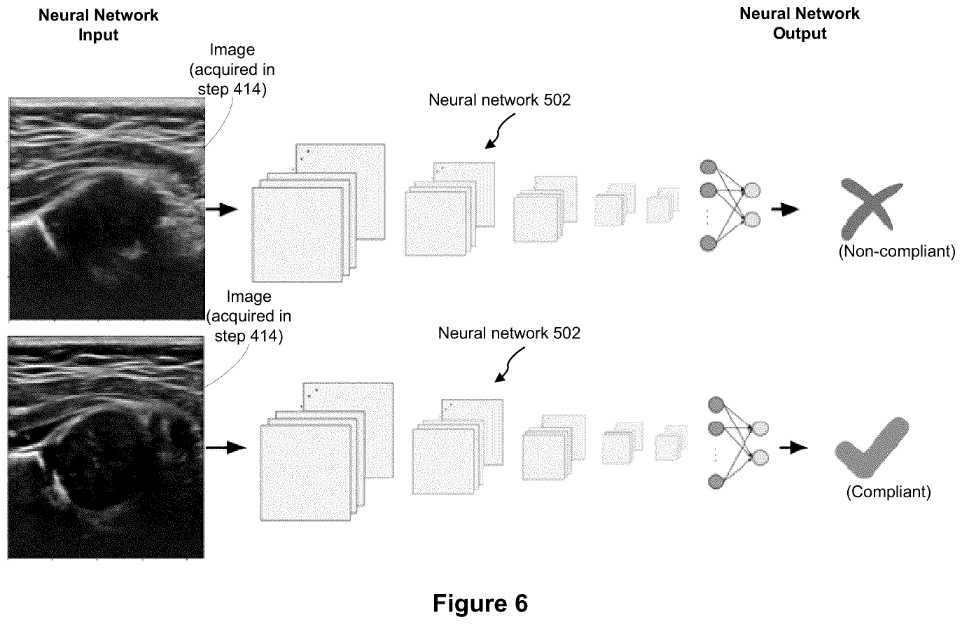

FIG. 6 illustrates an exemplary process for automatically assessing the quality of an ultrasound image and/or assigning an image quality score to an ultrasound image, in accordance with some embodiments.

In some embodiments, the ultrasound image that is acquired in step 414 is provided as an input to a trained neural network 502, such as a convolutional neural network (CNN), which has been trained to determine whether the image complies with the set of clinical requirements corresponding to the selected scan type. The output of this network may be one of n classes. FIG. 6 illustrates a non-limiting example where n is 2, and where the output of the trained neural network 502 is a binary output such as “compliant” or “non-compliant.” A compliant image is one that meets the image quality requirements corresponding to the scan type (see step 408), whereas a non-compliant image is one that does not meet at least one clinical requirement. In some embodiments, the image that is acquired in step 414 is provided as an input to a convolutional neural network (CNN) that is trained to output a real number (e.g., from 0 to 1, 0 to 100%, etc.) that indicates a proportion (e.g., a percentage) of the requirements that the image meets. In some embodiments, the image that is acquired in step 414 is provided as an input to a convolutional neural network (CNN) that is trained to output integer grades (e.g., from 1 to 5) that indicates a range of a proportion of the requirements that the image meets (optionally, more important requirements contribute more to the grade, if met). In some embodiments, the neural network is configured (e.g., trained) to provide an indication as to which individual requirements are met and which ones are not. As disclosed herein, a neural network is used as a nonlimiting example. Other machine learning, computer vision, image processing, and/or artificial intelligence techniques, such as rule-based systems, probabilistic models, template matching, attribute-based algorithms (e.g., decision trees, random forests, regression, support vector machines, etc.), can be used to implement the quality assessment of ultrasound images based on training.

In some embodiments, the neural network is trained by a training data set that includes a set of p images that have been determined as compliant, e.g., by a human expert, and a set of q images that have been labeled as non-compliant, e.g., by a human expert. When the number of classes n is more than two, the training data set comprises a set of images for each class to be identified. Each image is then input to the convolutional neural network, which includes a set of convolutional layers that is optionally followed by pooling, batch-normalization, dropout, dense, or activation layers. The output of the selected architecture is a vector of length n, where n is the number of classes to be identified. Each entry in the output vector is interpreted as the computed probability of belonging to each of the n classes. The output vector is then compared with a ground truth vector, which contains the actual probability of belonging to each of the n classes. The distance between the output vector and the ground truth vector is then computed using a loss function. Common loss functions are cross-entropy and its regularized versions; however, there are many loss functions that can be used for this process. The loss function is then used to compute an update to the weights of the neural network. Common optimization methods to compute this update are gradient-based optimization methods, such as gradient descent and its variants. The process of computing the loss and updating the weights is performed iteratively until a predetermined number of iterations is completed, or until a convergence criterion is met. In the case where the neural network is configured to output a real number representing the percentage of requirements that are being currently met by the acquired image. One possible implementation of this approach is to create a set of binary classifiers like the one described above. One binary classifier is trained for each clinical requirement, and the percentage of classifiers with a positive output is then computed. Images with enough quality then enter into the acquisition phase 404.

Referring now to step 420 of the workflow 400 in FIG. 4, in some embodiments, if the image quality requirements (e.g., clinical requirements corresponding to the scan type) are met, or if the image is classified as compliant, the workflow 400 proceeds to step 432, where the ultrasound probe enters the acquisition phase 404. In step 432, the ultrasound probe is automatically set to full-power mode (e.g., without any user intervention). In some embodiments, the ultrasound device generates an alert indicating that the quality requirements for the present scan is met, and that the acquisition phase is about to be started. This alert helps the operator to take notice of the current probe position and pose and start performing the scan in accordance with the present probe position and pose (e.g., maintaining the present position and pose, and/or starting the sweep from the present position and pose). In some embodiments, in the full-power mode, the computing device causes the ultrasound probe to activate all the available transducers in the ultrasound probe to acquire a high-quality image or a sequence of high quality images. In some embodiments, in the full-power mode, the computing device causes the ultrasound probe to activate another subset of all the available transducers (e.g., more than the subset of transducers corresponding to the low-power mode) to acquire a high-quality image.

In some embodiments, the computing device causes the ultrasound probe to remain in the full-power mode until the scan is finished. For example, as illustrated in steps 434, 436, 438, 440, 442, and 444 of the workflow 400, the ultrasound probe acquires an ultrasound image while operating in full-power mode. The image(s) that are acquired in the full-power mode are stored in an acquisition buffer of the ultrasound device. In some embodiments, when the ultrasound probe has finished scanning, the set of acquired images are saved (e.g., as ultrasound scan data 286 or ultrasound scan data 382). In some embodiments, when the computing device determines that the ultrasound probe has not finished scanning, the computing device causes the ultrasound scan to repeat steps 434, 436, and 438, until the ultrasound probe has completed the scan of the target anatomical structure, the set of images are then saved (e.g., as ultrasound scan data 286 or ultrasound scan data 382).

FIG. 4 also illustrates that in some embodiments, if not all of the image quality requirements are met (step 422), or if the ultrasound image that is acquired in the low-power mode in step 414 is classified as non-compliant, the computing device adds (424) the image to (e.g., stores the image in) an exploration buffer (e.g., in the memory 306).

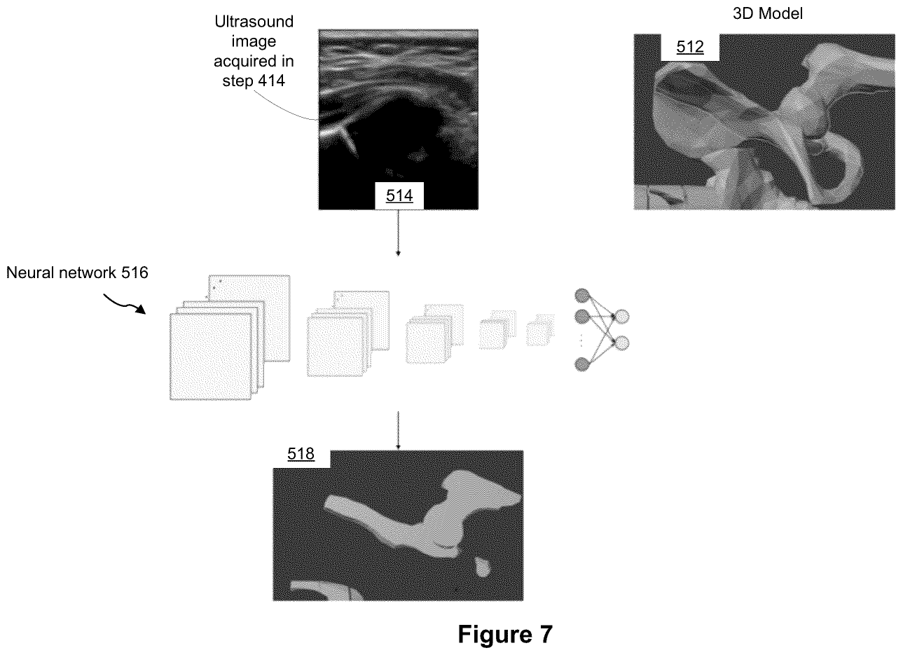

In some embodiments, the computing device uses the image (or multiple images) in the exploration buffer in at least one of two ways: (1) to provide guidance (step 426) to the user on how to position the probe to acquire a higher-quality image, or (2) to adjust the percentage of the transducers that are active in the ultrasound probe. These two approaches are described in greater detail with respect to FIGS. 7, 8, 9, 10, and 11.

FIG. 7 illustrates an exemplary process for providing guidance on positioning an ultrasound probe, in accordance with some embodiments.

Recall that in step 410 of the workflow 400, the computing device retrieves an atlas of anatomical structures of interest. In some embodiments, the atlas includes a 3D model of an anatomical structure of interest, such the 3D model 512 in FIG. 7. In some embodiments, given a 3D model of an anatomical structure of interest (e.g., 3D model 512) and an ultrasound image (e.g., image 514 in FIG. 7), the computing device determines (e.g., identify) (e.g., via an algorithm or a machine learning model) an anatomic plane 518 corresponding to an anatomical structure that is currently acquired by the ultrasound probe.

In some embodiments, the computing device determines an anatomic plane corresponding to an anatomical structure whose image is currently acquired by the ultrasound probe using a trained neural network 516. In some embodiments, the trained neural network 516 is a trained CNN. In some embodiments, the trained neural network 516 is configured to output a point in 6-dimensional space indicating the (x, y, z) position and rotations in the x-, y-, and z-axis with respect to the 3D model of the anatomical structure of interest. In some embodiments, the trained neural network 516 can output the angles of the imaging plane in the x-y, y-z, and x-z direction, as well as the distance of the plane to the origin of the 3D model. In some embodiments, the output includes guidance in terms of the probe position and pose relative to a desired position and pose of the probe to acquire a high quality image.

In some embodiments, the trained neural network 516 in FIG. 7 is similar to the trained neural network 502 in FIG. 6, but instead of giving, as an output, a vector representing probabilities, the trained neural network 516 can output a 6-dimensional vector with real values. Furthermore, the loss function might be a weighted sum of squared errors or any other loss function suitable for real-valued vectors that are not constrained to be probabilities.

In some embodiments, the computing device determines an anatomic plane corresponding to an anatomical structure whose image is currently acquired by the ultrasound probe by partitioning the angle-distance space into discrete classes, and then using a trained neural network (e.g., neural network 516, a CNN, etc.) that outputs the class of the input image. In some embodiments, the computing device includes (or is communicatively connected with) a bank of images (e.g., a database of images, such as labeled images 392 in the database 380) that has been labeled with their relative positions. The computing device identifies an image in the bank of images that is “closest” to the input image. Here, closest refers to determining the image that minimizes a distance function between the input image and every image in the bank.

In some embodiments, the computing device identifies imaging planes corresponding to respective (e.g., successive) ultrasound images in the exploration buffer and estimates (e.g., predicts), based on the identification, the trajectory that the ultrasound probe is following and what the next frame(s) will look like. In some embodiments, the computing device estimates the trajectory using temporal models that map an image into a point in a “probe-position space”, and then tracks the position of these points in the probe-position space over time. Models with this capability include Kalman Filters and similar probabilistic graphical models, recurrent neural networks, or reinforcement learning frameworks. The probe-position space can be determined, for example, by the spatial coordinates (e.g., x-, y-, and z-coordinates) and the three rotational angles in the x-, y-, and z-axes.

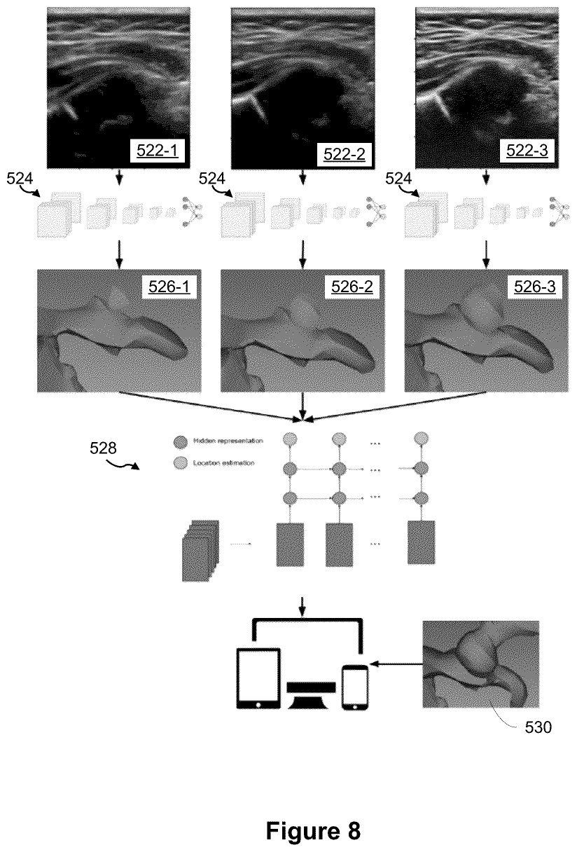

FIG. 8 illustrates an exemplary process for providing guidance to obtain an ultrasound image that meets image quality requirements, in accordance with some embodiments. In this example, the exploration buffer includes a series of ultrasound images (e.g., images 522-1, 522-2, and 522-3) that are acquired successively by an ultrasound probe. The images are used as inputs to a trained neural network 524, which is configured to output a representation 526 (e.g., a 3D representation) of an anatomical structure (e.g., a bone, a target organ, etc.) corresponding to the ultrasound image 522.

In some embodiments, the computing device computes a respective distance between a respective image 522 in the probe-position space and a desired image (e.g., an optimal image) (e.g., that is retrieved in steps 408 and 410) in a probe-location space, and determines, based on the computation, a sequence of steps that will guide the user to move (e.g., translate, rotate, and/or tilt) the probe in one or more directions and/or by various amounts to acquire the desired image. In some embodiments, the computing device causes the sequence of steps or instructions to be displayed on a display device that is communicatively connected with the ultrasound probe. In some embodiments, the guidance is dynamically updated in accordance with new images that are acquired after the probe position and pose is adjusted in accordance with initial step(s) of the guidance.

In some embodiments, depending on the distance between the current ultrasound image and the desired image, the computing device adjusts the percentage of the transducers that are activated. For example, the computing device activates (or causes activation of) a higher percentage and/or higher density of transducers when the distance between the current ultrasound image and the desired image decreases. The mapping (e.g., correlation) between the distance in the probe-position space and a percentage and/or density of active transducers can be implemented using regression models and their variants. Alternatively, a model that directly maps an image to a percentage and/or density of active transducers can be constructed using a combination of convolutional neural networks and recurrent neural networks. Referring again to FIG. 8, in this example, the images 522 are ultrasound images of the hip (although they can be of any anatomical structure of interest, such as the heart, lung, etc.). The images 522 are input into a neural network 524 to identify the anatomical part that is currently being imaged. The representations 526-1, 526-2, and 526-3 correspond to respective planes of the 3D anatomical structure (e.g., the hip) that is being displayed. The femoral head is not fully visible in the representation 526-1, whereas it is fully visible in the representation 526-3. The goal here is to obtain an image acquired from the plane denoted by representation 530. FIG. 8 also illustrates the representations 526 can be used as inputs to a trained recurrent neural network 528 that is configured to output a percentage and/or density of transducers to be activated to obtain an ultrasound image that meets the image quality requirements. The ultrasound probe activates (or is caused to activate) a larger number and/or density of transducers when the acquired image is closer to the target image. The exact number of transducers to be activated is computed by the recurrent neural network 528, which receives a sequence of representations 526 (e.g., imaging planes) and outputs the percentage of transducers to be activated.

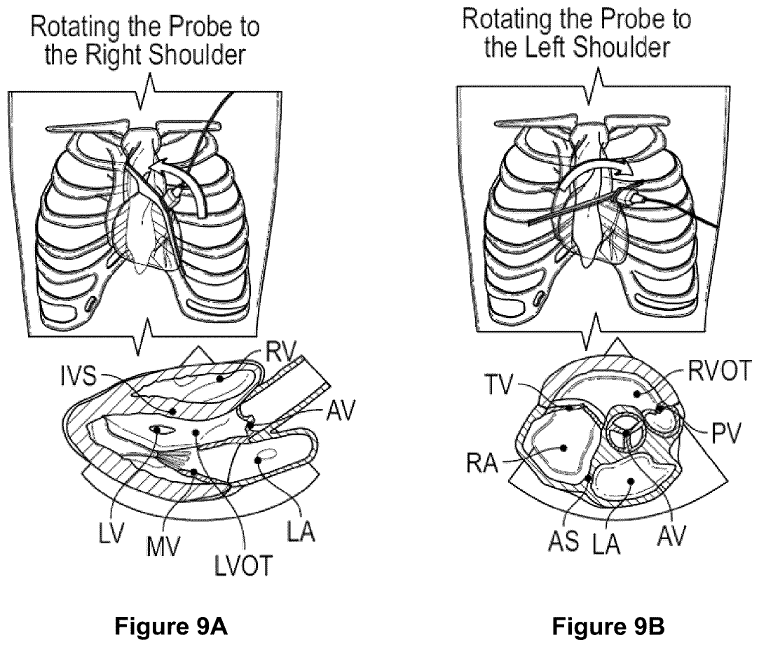

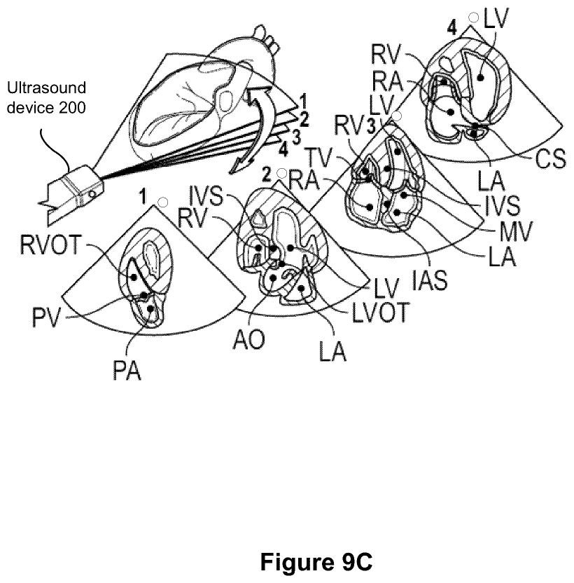

In some embodiments, instead of computing a distance between the current image in the probe-position space and a desired image, the computing device classifies the current image as one of n possible classes. FIGS. 9A to 9C illustrate changes in projected images of a heart with changes in ultrasound probe positioning, in accordance with some embodiments. For example, FIGS. 9A and 9B illustrate how a 2D projected image (e.g., an ultrasound image) of the heart changes as the ultrasound probe is rotated to the right shoulder in FIG. 9A and rotated to the left shoulder in FIG. 9B. FIG. 9C illustrates how changing a tilt and/or position of an ultrasound probe can lead to different planes of the heart to be imaged. Because the views of the anatomical planes are well-known in medical literature, it is possible to use a convolutional neural network to learn a classifier that identifies if the current view corresponds to, for example, view 1, view 2, view 3, or view 4 in FIG. 9C. Using FIG. 9C as an example, if a user is interested in acquiring view 3, the computing device can activate more transducers when the current image corresponds to view 2 than when the current image corresponds to view 1. Similarly, a convolutional neural network can also be trained to identify (e.g., recognize) images for probe rotation, such as the images shown in FIGS. 9A and 9B. Alternatively, in some embodiments, the percentage of transducers active at a given time can be a function of the number of movements needed to get from the current view to the desired view. In some embodiments, the computing device provides visual, audial, and haptic feedback to the user, to indicate the changes of the percentage and/or density of transducers that are activated.

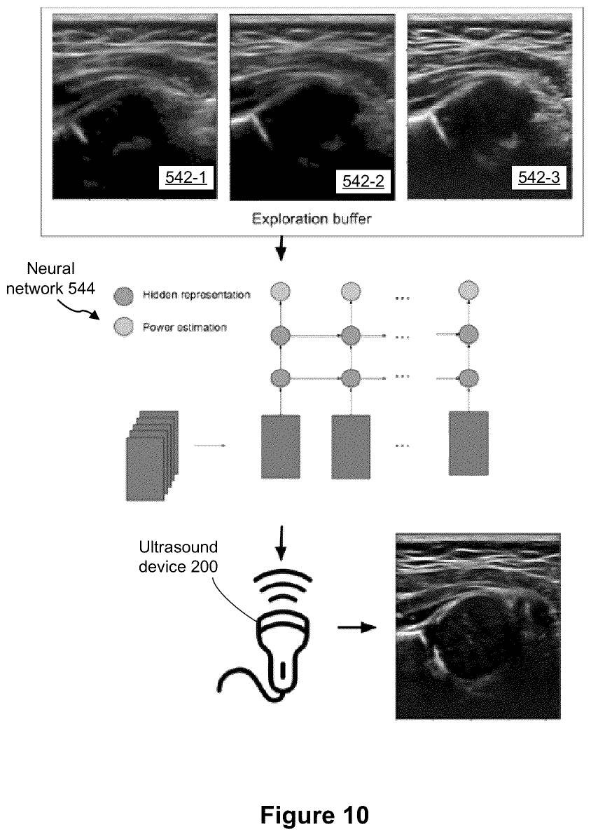

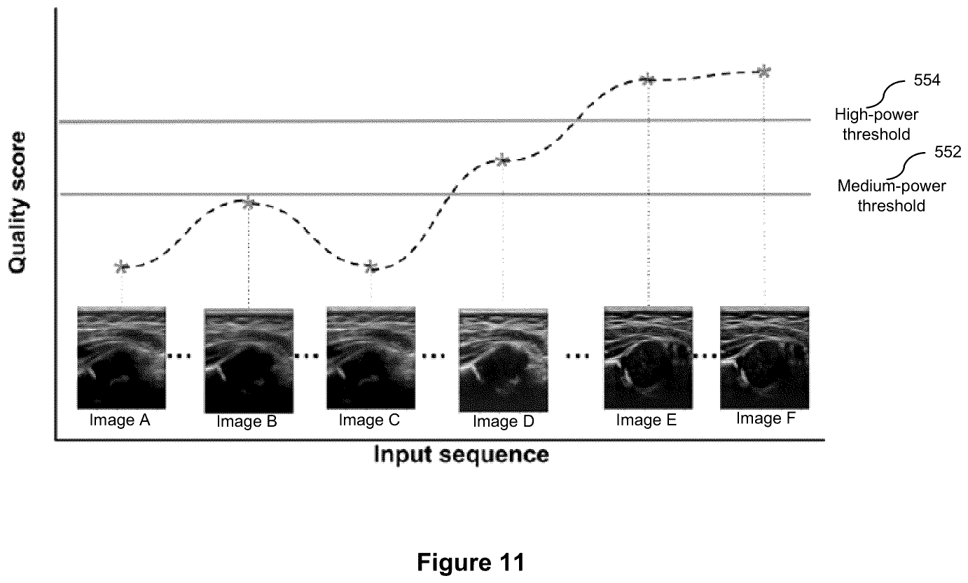

FIGS. 10 and 11 illustrate exemplary processes for adjusting the percentage and/or density of the transducers that are active in the ultrasound probe to acquire subsequent ultrasound images, in accordance with some embodiments.

FIG. 10 illustrates an exemplary process for providing user guidance to acquire a higher quality ultrasound image, in accordance with some embodiments. In some embodiments, after analyzing ultrasound images (e.g., images 542-1, 542-2, and 542-3) in the exploration buffer, the computing device adjusts the power of the ultrasound probe (or causes the ultrasound probe to adjust its power) by adjusting (e.g., increasing) the percentage and/or density of transducers that are activated in the ultrasound probe. The computing device provides guidance to a user of the ultrasound to acquire a higher quality image with the higher number and/or density of activated transducers. One possible implementation of this method is via a recurrent neural network (RNN) (e.g., neural network 544, FIG. 10) that maps an ultrasound image to a percentage (or distribution of) of active transducers (see also FIG. 8 and the accompanying description). A similar algorithm might be implemented using convolutional neural networks.

Alternatively, in some embodiments, the goal of providing user guidance can be analogized to solving a kinematics problem. For example, the sequence of ultrasound images that have been acquired (and stored in the exploration buffer) can be mapped to a six-dimensional space that includes the x-, y-, and z-axes of the probe and the angles with respect to each of these axes. This mapping can be implemented using a convolutional neural network that maps an ultrasound image in the exploration buffer to a six-dimensional vector, or by comparing the current view with a database that contains a set of images with different views and their corresponding coordinates (e.g., atlas 386 or labeled images 392), and then assigning to the current view the coordinates of the closest image. Then, the computing device can identify the changes in the x-, y-, and/or z-coordinates and/or angles (e.g., an angle measured with respect to the x-y plane, an angle measured with respect to the y-z plane, and/or an angle measured with respect to the z-x plane) that will decrease the distance between a six-dimensional vector representation of a current ultrasound image (e.g., current view acquired by the ultrasound probe) and a six-dimensional vector representation of the ground truth. One possible way of computing such changes is using a process known as visual servoing. Visual servoing assumes that the ultrasound probe is a mechanism with six degrees of freedom. It then estimates the changes in each degree of freedom that decreases the distance between the current and the target position. By using Broyden's method, along with a local linear model, the computing device can iteratively compute the sequence of steps that the user needs to execute to move the probe to the desired position to obtain a desired ultrasound image of the target anatomical structure. In some embodiments, the computer device outputs visual, audial, and haptic feedback to the user to guide the user to move the probe to the desired position and pose.

FIG. 11 illustrates an input sequence of ultrasound images and their respective quality scores, in accordance with some embodiments.

In some embodiments, the computing device tracks the computed quality scores of the ultrasound images as the images are successively acquired over time (e.g., in the exploration phase, and optionally in the acquisition phase), and adjusts the percentage of transducers that are activated according to a detected trend in the image quality scores. For example, in some embodiments, the computing device increases (or causes to be increased) the number of transducers that are activated in an ultrasound probe after the quality score crosses a medium-power threshold score 552. In some embodiments, the number of transducers that are activated is again increased after the quality is higher than a high-power threshold score 554.

Referring again to the workflow 400 in FIG. 4, in some embodiments, the process of providing guidance to obtain an image that meets image quality requirements is repeated (e.g., as a loop) until the ultrasound probe enters the acquisition phase, as shown in steps 414, 416, 418, 422, 424, 426, 428, and 430 in FIG. 4.

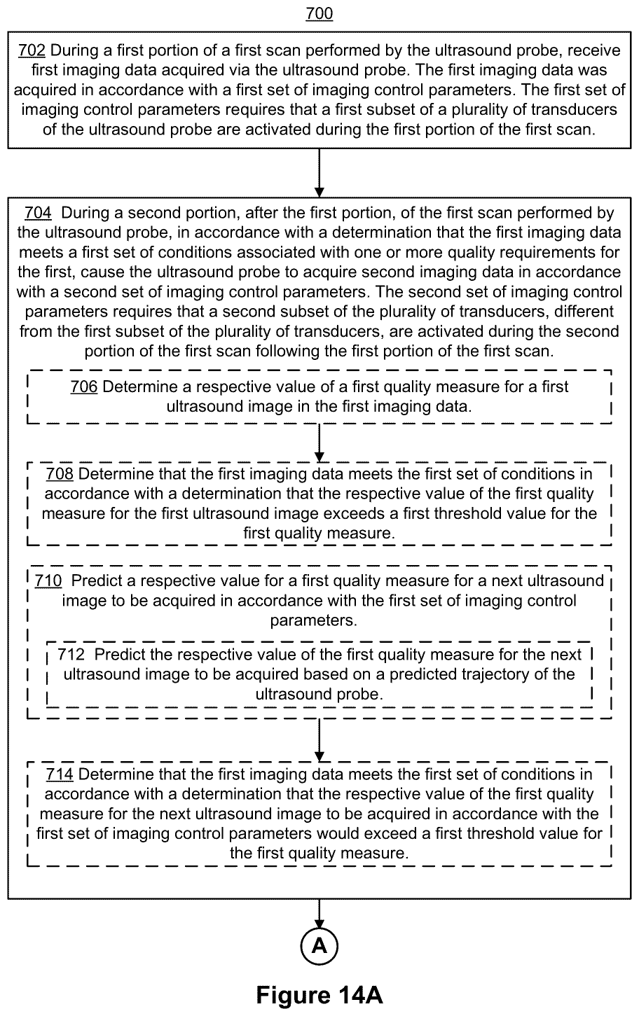

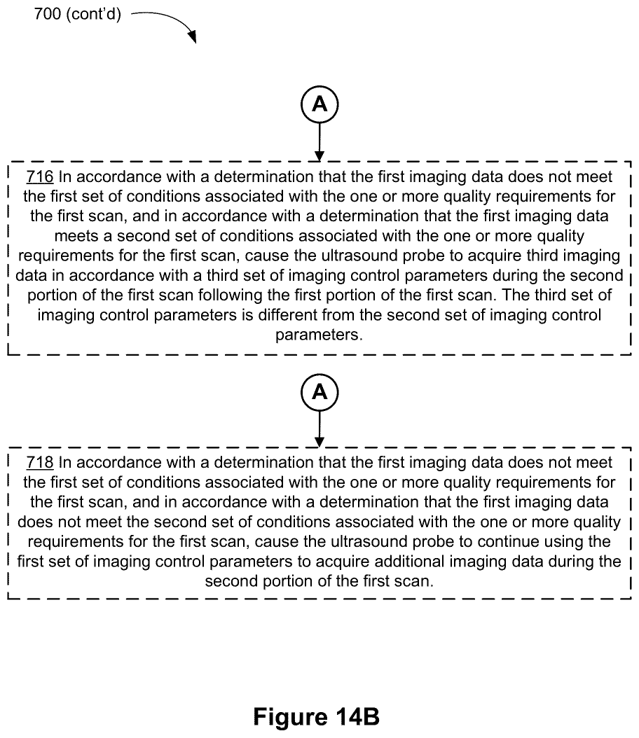

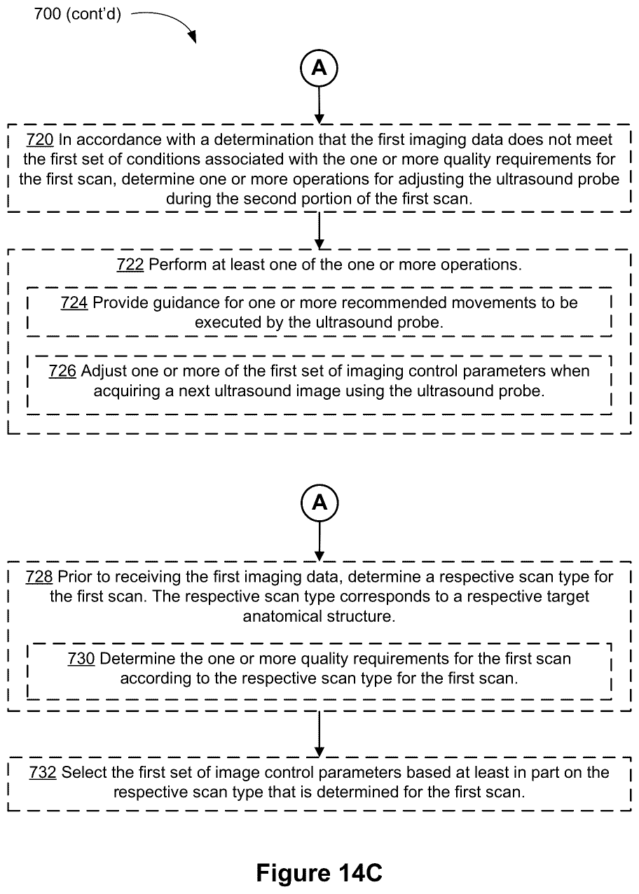

FIGS. 14A to 14C illustrates a flowchart diagram for a method 700 of controlling an ultrasound probe (e.g., to manage heat generation, power consumption, imaging fidelity, and address other requirements and considerations of ultrasound imaging using an ultrasound probe), in accordance with some embodiments. In some embodiments, the ultrasound probe (e.g., ultrasound device 200) is a handheld ultrasound probe, or an ultrasound scanner with an automatic probe. In some embodiments, the method 700 is performed at a computing device (e.g., computing device 130 or computing device 300) that includes one or more processors (e.g., CPU(s) 302) and memory (e.g., memory 306). For example, in some embodiments, the computing device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system.

During a first portion of a first scan performed by the ultrasound probe (e.g., during an exploration phase 402 of the scan, during an initial portion of the exploration phase of the scan, and/or during a sweep of the ultrasound probe across a portion of a patient's body), the computing device receives (702) first imaging data acquired via the ultrasound probe. The first imaging data was acquired in accordance with a first set of imaging control parameters (e.g., device settings 282 or imaging control parameters 388). In some embodiments, the first set of imaging control parameters correspond to the low-power mode. The first set of imaging control parameters requires that a first subset of a plurality of transducers (e.g., transducers 220) of the ultrasound probe are activated during the first portion of the first scan. For example, the first subset of the plurality of transducers can include one of: while less than half of all of the transducers are activated; while more than half, but less than all, of the plurality of transducers of the ultrasound probe are activated; while some, less than half, of a plurality of transducers of the ultrasound probe are not activated; or while all of the plurality of transducers of the ultrasound probe are activated.

In some embodiments, the first imaging data includes a first set of ultrasound images. In some embodiments, the first imaging data includes images that have been preprocessed to meet the input requirement of an AI image assessment algorithm, and/or images or raw data that are directly generated by the ultrasound probe. In some embodiments, the first imaging data include sequential images acquired by the ultrasound probe during a respective scan or a sweep (e.g., during a first portion of the scan or sweep) using the ultrasound probe.

In some embodiments, the first set of imaging control parameters includes one or more of: a number of transducers that are activated, a power consumption threshold of the probe, an imaging frame rate, a scan speed, and other scan parameters that control the power consumption, heat generation rate, and/or processing load of the probe.

FIG. 14A illustrates that, during a second portion, after the first portion, of the first scan performed by the ultrasound probe, in accordance with a determination that the first imaging data meets a first set of conditions associated with one or more quality requirements for the first scan, the computing device causes (704) (e.g., through one or more instructions, signals, machine commands, inputs, and/or operations implemented using software, hardware, firmware, and/or other control means) the ultrasound probe to acquire second imaging data in accordance with a second set of imaging control parameters. The second set of imaging control parameters requires that a second subset of the plurality of transducers, different from the first subset of the plurality of transducers, are activated during the second portion of the first scan following the first portion of the first scan.

For example, in some embodiments, the first set of conditions associated with one or more quality requirements for the first scan includes one or more of: a condition that the first imaging data includes one or more newly acquired images that meet one or more threshold quality scores, a condition that the first imaging data includes one or more newly acquired images that correspond to one or more anatomical planes that match a desired anatomical plane of a target anatomical structure, a condition that the first imaging data includes one or more newly acquired images include one or more landmark/features (or a combination of landmarks/features), a condition that the first imaging data includes one or more newly acquired images that include a feature having a particular dimension, a condition that the first imaging data supports a prediction that an image meeting one or more requirements would be acquired in the next one or more image frames, and a condition that the first imaging data supports a prediction that a first change (e.g., an increase by a percentage, or number) in the number of transducer used would support an improvement in the quality score of an image acquired in the next one or more image frames, and/or other analogous conditions).

In some embodiments, the second subset of the plurality of transducers includes the first subset of the plurality of transducers and additional transducers that were not included in the first subset of the plurality of transducers. In some embodiments, the second subset of the plurality of transducers includes fewer than the first subset of the plurality of transducers, and/or includes at least some transducers that were not included in the first subset of transducers.

In some embodiments, the second subset of the plurality of transducers consists of all the transducers of the ultrasound probe.

In some embodiments, the second subset of the plurality of transducers correspond to the full-power mode of the ultrasound probe (see, e.g., step 432, FIG. 4).

In some embodiments, the computing device causes the ultrasound probe to acquire the second imaging data in accordance with the second set of imaging control parameters until completion of the first scan.

With continued reference to FIG. 14A, in some embodiments, determining whether the first imaging data meets the first set of conditions includes determining (706) a respective value of a first quality measure (e.g., a quality score; a percentage of requirements that are met or unmet; a number of image landmarks identified; one of a plurality of quality classes, such as best, good, satisfactory, poor, and so on) for a first ultrasound image in the first imaging data. In some embodiments, the computing device determines that the first imaging data meets the first set of conditions in accordance with a determination (708) that the respective value of the first quality measure for the first ultrasound image exceeds a first threshold value for the first quality measure.

In some embodiments, the first quality measure comprises a quality score. In some embodiments, the quality score comprises a binary classification (e.g., positive or negative; 0 or 1, compliant or non-compliant, etc.). In some embodiments, the quality score is a real number (e.g., that ranges from 0 to 1, 0 to 1.0, etc.). In some embodiments, the quality score is values expressed as a ratio, or as a percentage.

In some embodiments, the first set of conditions associated with the one or more quality requirements for the first scan includes a first condition that the first imaging data (or at least one ultrasound image in the first imaging data) satisfies a first threshold score.

In some embodiments, the computing device assigns a respective value of the first quality measure to the first ultrasound image automatically and without user intervention.

In some embodiments, assigning a respective value of the first quality measure to the first ultrasound image includes using the first imaging data (or using the first ultrasound image) as an input to a trained neural network (e.g., a convolutional neural network) (e.g., neural network 502, FIG. 6) that is configured to generate an output indicating whether the first imaging idea satisfies the first threshold score.

In some embodiments, the output of the trained neural network is a binary output consisting of: (i) a first output (compliant output) indicating that the first imaging data meets the first set of conditions associated with one or more quality requirements for the first scan; and (ii) a second output (non-compliant output) indicating that the first imaging data does not meet the first set of conditions associated with one or more quality requirements for the first scan. This is illustrated in, e.g., FIG. 6.

In some embodiments, the output of the trained neural network comprises a real number (e.g., 0-100, or 0.0-1.0) corresponding to a proportion (percentage) of the first imaging data satisfying the one or more quality requirements for the first scan.

In some embodiments, the computing device predicts (710) a respective value for a first quality measure (e.g., a quality score; a percentage of requirements that are met or unmet; a number of image landmarks identified; one of a plurality of quality classes, such as best, good, satisfactory, poor, and so on) for a next ultrasound image to be acquired in accordance with the first set of imaging control parameters.

In some embodiments, the computing device determines (714) that the first imaging data meets the first set of conditions in accordance with a determination that the respective value of the first quality measure for the next ultrasound image to be acquired in accordance with the first set of imaging control parameters would exceed a first threshold value for the first quality measure.

In some embodiments, the first imaging data includes a plurality of images. In some embodiments, in accordance with a determination that the first imaging data does not meet the first set of conditions associated with one or more quality requirements for the first scan, the computing device determines, for each image in the plurality of images of the first imaging data, a respective anatomic plane of a first target anatomical structure corresponding to the first scan, so as to obtain a plurality of anatomic planes of the first target anatomical structure.

In some embodiments, the computing device predicts (712) the respective value of the first quality measure for the next ultrasound image to be acquired based on a predicted trajectory of the ultrasound probe.

For example, in some embodiments, the computing device determines (e.g., estimates) a trajectory of the ultrasound probe based on the plurality of anatomic planes for the first target anatomical structure. The computing device can estimate this trajectory using temporal models that map an image into a point in a “probe-position space” and tracking the position of these points in the probe-position space over time. Models with this capability include Kalman Filters and similar probabilistic graphical models, recurrent neural network, or reinforcement learning frameworks. The probe-position space can be determined, for example, by 3 spatial coordinates (e.g., x-, y-, and z-coordinates) and the 3 rotational angles in the x, y, and z-axes. In some embodiments, the computing device predicts a quality score for a subsequent image frame acquired by the ultrasound probe in accordance with the determined trajectory.

Referring now to FIG. 14B, in some embodiments, in accordance with a determination that the first imaging data does not meet the first set of conditions associated with the one or more quality requirements for the first scan (e.g., the first imaging data does not support a prediction that the next image would meet the one or more quality requirements, and/or the first imaging data does not meet the one or more quality requirements) (e.g., step 422, FIG. 4), and in accordance with a determination that the first imaging data meets a second set of conditions associated with the one or more quality requirements for the first scan (e.g., the first imaging data support a prediction that increasing the number of transducers used would improve image quality with regard to the one or more quality requirements), the computing device causes (716) the ultrasound probe to acquire third imaging data in accordance with a third set of imaging control parameters during the second portion of the first scan following the first portion of the first scan, wherein the third set of imaging control parameters is different from the second set of imaging control parameters. In some embodiments, the third set of imaging control parameters requires a third subset of transducers, more than the first subset of transducers and fewer than the second subset of transducers, of the plurality of transducers of the ultrasound probe to be active during the second portion of the first scan.

In some embodiments, in accordance with a determination that the first imaging data does not meet the first set of conditions associated with the one or more quality requirements for the first scan (e.g., the first imaging data does not support a prediction that the next image would meet the one or more quality requirements, and/or the first imaging data does not meet the one or more quality requirements), and in accordance with a determination that the first imaging data does not meet the second set of conditions associated with the one or more quality requirements for the first scan (e.g., the first imaging data does not support a prediction that increasing the number of transducers used would improve image quality with regard to the one or more quality requirements), the computing device causes (718) the ultrasound probe to continue using the first set of imaging control parameters to acquire additional imaging data during the second portion of the first scan.

Referring to FIG. 14C, in some embodiments, in accordance with a determination that the first imaging data does not meet the first set of conditions associated with the one or more quality requirements for the first scan, the computing device determines (720) one or more operations for adjusting the ultrasound probe during the second portion of the first scan. The computing device performs (722) at least one of the one or more operations.

For example, in some embodiments, in accordance with a determination that the first imaging data does not meet the first set of conditions associated with one or more quality requirements for the first scan, the computing device determines, from the first imaging data, a first anatomic plane of a first target anatomical structure corresponding to the first imaging data. In some embodiments, after the computing device determines the first anatomic plane, the computing device generates one or more recommendations for adjusting at least one of: a position of the ultrasound probe or an orientation of the ultrasound probe, so that subsequent imaging data acquired by the ultrasound probe corresponds to a second anatomic plane of a first target anatomical structure.

In some embodiments, the computing device determines the first anatomic plane of the first target anatomical structure by using the first imaging data as input data to a trained neural network that is configured to generate, as output data, at least one of: (i) one or more angles of the anatomic plane of the first target anatomical structure relative to one or more reference planes of a model of the first target anatomical structure and (ii) a distance between the anatomic plane and an origin of the model of the first target anatomical structure. In some embodiments, the one or more angles are relative to one or more reference planes (e.g., the one or more angles include a first angle in the x-y direction, determined relative to a reference x-y plane; include a second angle in the y-z direction, determined relative to a reference y-z plane, and include a third angle in the x-z direction, determined relative to a reference x-z plane. In some embodiments, the neural network outputs a point in 6-dimensional space indicating the (x, y, z) position and rotations in the (x, y, z)-axis with respect to the 3D model of the anatomical structure of interest.

In some embodiments, the computing device determines the first anatomic plane of the first target anatomical structure by using the first imaging data as input data to a trained neural network that is configured to generate, as output data, one or more classes corresponding to the first imaging data. For example, in some embodiments, the one or classes are determined based on one or more angles of the anatomic plane of the first target anatomical structure relative to one or more reference planes of a model of the first target anatomical structure.

In some embodiments, the computing device determines the first anatomic plane of the first target anatomical structure by comparing the first imaging data with a repository (e.g., database) of reference images of the first target anatomical structure labeled with positional information (e.g., relative positional information, including angles relative to one or more reference planes) (e.g., labeled images 392) according to a first criterion, and selecting, in accordance with the comparison, a first reference image from the repository of reference images that satisfies the first criterion.

With continued reference to FIG. 14C, in some embodiments, performing at least one of the one or more operations includes providing (724) guidance (e.g., via speech, sound, images, animations, and/or other audio, visual, and/or haptic feedback mechanisms) for one or more recommended movements (e.g., direction, distance, path, title angles, and/or other movements) to be executed by (e.g., using) the ultrasound probe (e.g., automatically, or under the direction and/or manual control of a technician).

In some embodiments, performing at least one of the one or more operations includes adjusting (726) one or more of the first set of imaging control parameters (e.g., frequency, phase, duration, power, direction, plane, and/or other parameters) when acquiring a next ultrasound image using the ultrasound probe. The adjusting can occur automatically (without user intervention) or under the direction and/or manual control of a technician.

In some embodiments, prior to receiving the first imaging data, the computing device determines (728) (e.g., based on user selection, based on previous scan type, based on institution type, based on most recent scan type, based on frequently used scan type, based on operator ID, based on medical record, based on scan order information, and/or based on other past and present contextual data) a respective scan type for the first scan. The respective scan type corresponds to a respective target anatomical structure.

In some embodiments, the computing device determines the one or more quality requirements (e.g., image quality requirements 288) for the first scan according to (730) the respective scan type for the first scan. In some embodiments, the one or more quality requirements for the first scan include one or more clinical requirements corresponding to the first scan type.

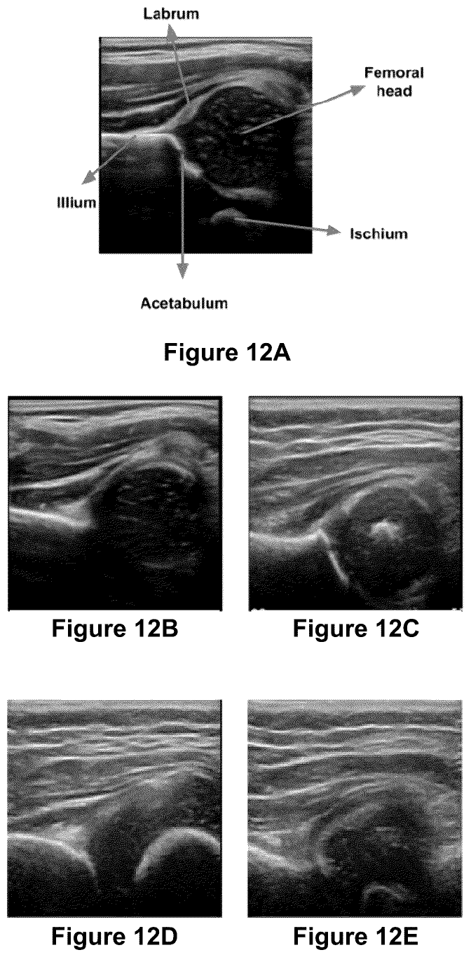

Examples of scan types include a transversal view of the hip, a 4-chamber echocardiogram, a longitudinal scan of the lung, and/or another type of ultrasound scan target. Generally speaking, each scan type has its own clinical requirements. For example, the clinical requirements for an ultrasound image of a hip to determine the presence of hip dysplasia requires presence of the labrum, ischium, the midportion of the femoral head, flat and horizontal ilium, and absence of motion artifact. FIG. 12A is an exemplary ultrasound image of a hip that meets all the clinical requirements for determining the presence of hip dysplasia. By contrast, FIGS. 12B, 12C, 12D, and 12E are exemplary ultrasound images of the hip which do not meet the clinical requirements for determining the presence of hip dysplasia, because the acetabulum and the ischium are not visible in FIG. 12B, the ischium is not visible in FIG. 12C, and most of the elements are not visible in FIGS. 12D and 12E. In this example, the images in FIG. 12D and 12E are acquired when the ultrasound probe is operating in a low-power setting, while the images in FIGS. 12B and 12C are acquired when the ultrasound probe is operating in a medium-power setting. According to some aspects of the present disclosure, the ultrasound images corresponding to FIGS. 12D to 12E should be acquired while the ultrasound probe is operating in a low-power setting.

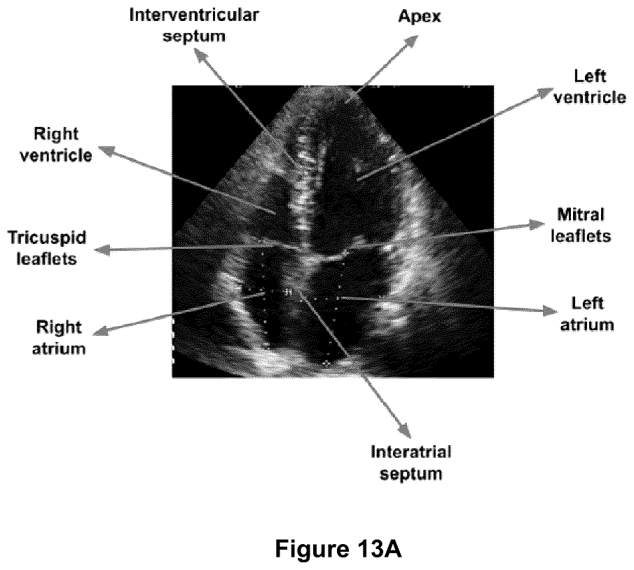

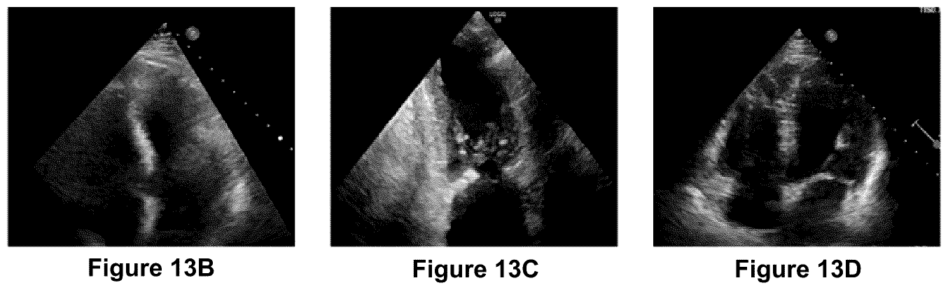

As another example, the clinical requirements for an echocardiography 4-chamber apical view are: (i) a view of the four chambers (left ventricle, right ventricle, left atrium, and right atrium) of the heart, (ii) the apex of the left ventricle is at the top and center of the sector, while the right ventricle is triangular in shape and smaller in area, (iii) myocardium and mitral leaflets should be visible, and (iv) the walls and septa of each chamber should be visible. FIG. 13A illustrates an ultrasound image of the heart that meets the clinical requirements. By contrast, FIGS. 13B to 13D illustrate ultrasound images of the art that do not meet all of the clinical requirements. For example, in FIG. 13B, the four chambers of the heart are not clearly defined, and the mitral and tricuspid leaflets are not visible. In FIG. 13C, only the left ventricle and part of the left atrium are visible. The apex of the left ventricle lies outside of the image. In FIG. 13D, the tricuspid leaflets are not visible, and the walls of the left ventricle are not clearly visible.

In some embodiments, the computing device selects (732) the first set of image control parameters based at least in part on the respective scan type that is determined for the first scan.

In some embodiments, the first scan comprises a scan of a target anatomical structure. The computing device determines whether the first imaging data meets the first set of conditions by comparing the first imaging data with (e.g., a database of) generalized imaging data of the target anatomical structure (e.g., an atlas 290, or at atlas 386, or labeled images 392 of anatomical structure of interest). In some embodiments, the generalized imaging data of the target anatomical structure comprises three-dimensional imaging data of the target anatomical structure.

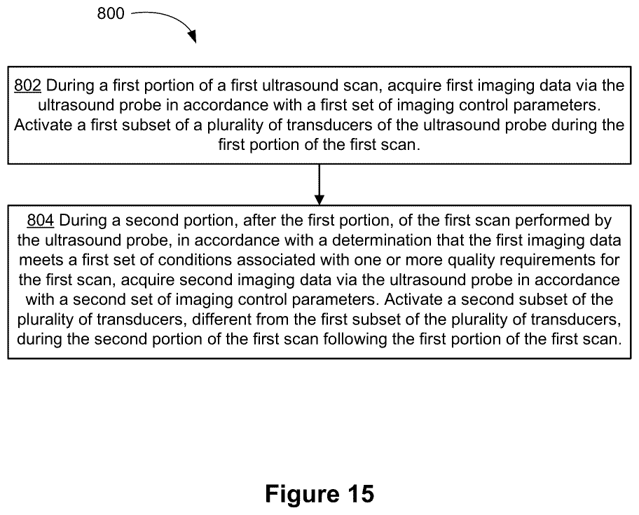

FIG. 15 illustrates a flowchart diagram for a method 800 for controlling an ultrasound probe (e.g., to manage heat generation, power consumption, imaging fidelity, and address other requirements and considerations of ultrasound imaging using an ultrasound probe) in accordance with some embodiments. The method 800 is performed at a computing device that includes one or more processors and memory. In some embodiments, the ultrasound probe comprises a handheld ultrasound probe or an ultrasound scanner. The ultrasound probe is communicatively connected to the computing device. In some embodiments, the computing device is a server or control console that is in communication with the ultrasound probe or is integrated into the ultrasound probe. Additional details of the method 800 can be found in FIGS. 1 to 14C and the accompanying descriptions, and are not repeated for the sake of brevity.

The computing device, during a first portion of a first ultrasound scan, acquires (802) first imaging data via the ultrasound probe in accordance with a first set of imaging control parameters, including activating a first subset of a plurality of transducers of the ultrasound probe during the first portion of the first scan.

In some embodiments, the first portion of the first ultrasound scan corresponds to during an exploration phase (e.g., exploration phase 402) of the scan, during an initial portion of the exploration phase of the scan, and/or during a sweep of the ultrasound probe across a portion of a patient's body.

In some embodiments, the first imaging data includes a first set of ultrasound images, images that have been preprocessed to meet the input requirement of an AI image assessment algorithm, and/or images or raw data that are directly generated by the ultrasound probe. In some embodiments, the first imaging data includes one or more images that are sequentially acquired by the ultrasound probe during a respective scan or a sweep (e.g., during a first portion of the scan or sweep) using the ultrasound probe.

In some embodiments, the first set of imaging control parameters includes: a number of transducers that are activated, power consumption threshold of the probe, an imaging frame rate, a scan speed, and/or other scan parameters that control the power consumption, heat generation rate, and./or the processing load of the ultrasound probe.

In some embodiments, activating the first subset of a plurality of transducers of the ultrasound probe includes activating more than half, but less than all, of the plurality of transducers of the ultrasound probe during the first portion of the first scan, while some, less than half, of the plurality of transducers of the ultrasound probe are not activated during the first portion of the first scan.

The computing device, during a second portion, after the first portion, of the first scan performed by the ultrasound probe, in accordance with a determination that the first imaging data meets a first set of conditions associated with one or more quality requirements for the first scan, acquires (804) second imaging data via the ultrasound probe in accordance with a second set of imaging control parameters, including activating a second subset of the plurality of transducers, different from the first subset of the plurality of transducers, during the second portion of the first scan following the first portion of the first scan.

In some embodiments, the first set of conditions associated with one or more quality requirements for the first scan includes: a condition that the first imaging data includes one or more newly acquired images that meet one or more threshold quality scores, a condition that the first imaging data includes one or more newly acquired images that correspond to one or more anatomical planes that match a desired anatomical plane of a target anatomical structure, a condition that the first imaging data includes one or more newly acquired images that include one or more landmark/features (or a combination of landmarks/features), a condition that the first imaging data includes one or more newly acquired images that include a feature having a particular dimension, a condition that the first imaging data supports a prediction that an image meeting one or more requirements would be acquired in the next one or more image frames, a condition that the first imaging data supports a prediction that a first change (e.g., an increase by a percentage, or number) in the number of transducer used would support an improvement in the quality score of an image acquired in the next one or more image frames, and/or other analogous conditions.

In some embodiments, the second subset of the plurality of transducers includes the first subset of the plurality of transducers and additional transducers that were not included in the first subset of the plurality of transducers. In some embodiments, the second subset of the plurality of transducers includes a fewer number of transducers than the first subset of the plurality of transducers. In some embodiments, the second subset of the plurality of transducers includes at least some transducers that were not included in the first subset of transducers. In some embodiments, the second subset of the plurality of transducers includes all of the plurality of transducers.

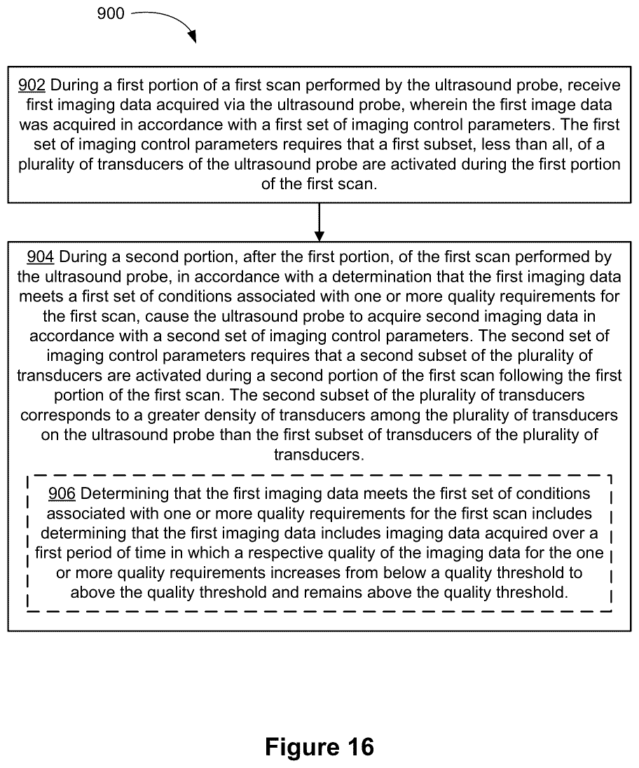

FIG. 16 illustrates a flowchart diagram for a method 900 of managing heat generation on an ultrasound probe (e.g., ultrasound device 200, or probe portion of ultrasound device 200) in accordance with some embodiments. The method 900 is performed at a computing device (e.g., computing device 300 that includes one or more processors (e.g., CPU(s) 302) and memory (e.g., memory 306). In some embodiments, the computing device is a server or control console that is in communication with the ultrasound probe or is integrated into the ultrasound probe. In some embodiments, the ultrasound probe comprises a handheld ultrasound probe or an ultrasound scanner. The ultrasound probe is communicatively connected to the computing device. Additional details of the method 900 can be found in FIGS. 1 to 15 and the accompanying descriptions, and are not repeated for the sake of brevity.

The computing device, during a first portion of a first scan performed by the ultrasound probe, receives (902) first imaging data acquired via the ultrasound probe. The first image data was acquired in accordance with a first set of imaging control parameters. The first set of imaging control parameters requires that a first subset, less than all, of a plurality of transducers of the ultrasound probe are activated during the first portion of the first scan.

In some embodiments, the first portion of the first scan corresponds to during an exploration phase of the scan, during an initial portion of the exploration phase of the scan, and/or during a sweep of the ultrasound probe across a portion of a patient's body.

In some embodiments, the first imaging data includes a first set of ultrasound images, images that have been preprocessed to meet the input requirement of an AI image assessment algorithm, and/or images or raw data that are directly generated by the ultrasound probe. In some embodiments, the first imaging data includes sequential images acquired by the ultrasound probe during a respective scan or a sweep (e.g., during a first portion of the scan or sweep) using the ultrasound probe.

In some embodiments, the first set of imaging control parameters includes a number of transducers that are activated, a power consumption threshold of the probe, an imaging frame rate, a scan speed, and/or other scan parameters that control the power consumption, heat generation rate, and processing load of the probe.

In some embodiments, the requirement that the first subset of the plurality of transducers are activated during the first portion of the first scan includes a requirement that more than half, but less than all, of the plurality of transducers of the ultrasound probe are activated during the first portion of the first scan, while some, less than half, of a plurality of transducers of the ultrasound probe are not activated during the first portion of the first scan. In some embodiments, the requirement that the first subset of the plurality of transducers are activated during the first portion of the first scan includes a requirement that at least a threshold percentage of all of the plurality of transducers of the ultrasound probe are activated.

The computing device, during a second portion, after the first portion, of the first scan performed by the ultrasound probe, in accordance with a determination that the first imaging data meets a first set of conditions associated with one or more quality requirements for the first scan, causes (904) (e.g., through one or more instructions, signals, machine commands, inputs, and/or operations implemented using software, hardware, firmware, and/or other control means) the ultrasound probe to acquire second imaging data in accordance with a second set of imaging control parameters. The second set of imaging control parameters requires that a second subset of the plurality of transducers are activated during a second portion of the first scan following the first portion of the first scan. The second subset of the plurality of transducers corresponds to a greater density of transducers among the plurality of transducers on the ultrasound probe than the first subset of transducers of the plurality of transducers.

In some embodiments, the first set of conditions associated with one or more quality requirements for the first scan includes one or more of: a condition that the first imaging data includes one or more newly acquired images that meet one or more threshold quality scores, a condition that the first imaging data includes one or more newly acquired images that correspond to one or more anatomical planes that match a desired anatomical plane of a target anatomical structure, a condition that the first imaging data includes one or more newly acquired images include one or more landmark/features (or a combination of landmarks/features), a condition that the first imaging data includes one or more newly acquired images that include a feature having a particular dimension, a condition that the first imaging data supports a prediction that an image meeting one or more requirements would be acquired in the next one or more image frames, a condition that the first imaging data supports a prediction that a first change (e.g., an increase by a percentage, or number) in the number of transducer used would support an improvement in the quality score of an image acquired in the next one or more image frames, and/or other analogous conditions.

In some embodiments, the second subset of the plurality of transducers includes the first subset of the plurality of transducers and additional transducers that were not included in the first subset of the plurality of transducers. In some embodiments, the second subset of the plurality of transducers includes all of the plurality of transducers.

In some embodiments, determining that the first imaging data meets the first set of conditions associated with one or more quality requirements for the first scan includes determining (906) that the first imaging data includes imaging data acquired over a first period of time in which a respective quality of the imaging data for the one or more quality requirements increases from below a quality threshold to above the quality threshold and remains above the quality threshold.

For example, FIG. 11 illustrates an input sequence of images (image A, image B, image C, image D, image E, and image F) and their respective quality scores. In the example of FIG. 11, there is an increase in quality score from image A to image B, a decrease in quality score from image B and image C, and successive increases in quality scores from image C to image D, then from image D to image E, and from image E to image F. In particular, image C has a quality score that is below a medium-power threshold 552 whereas image D has a quality score that is above the medium-power threshold 552. Images E and F have quality scores that are above a high-power threshold 554. In this example, the computing device can determine that the input sequence of images meets the set of conditions associated with one or more quality requirements because the quality score for increases below a quality threshold (e.g., below the medium-power threshold 552) in Image C to above the quality threshold and remains above the quality threshold for subsequent images D, E, and F.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

It will also be understood that, although the terms first, second, etc. are, in some instances, used herein to describe various elements, these elements should not be limited by these terms. These terms are only used to distinguish one element from another. For example, a first transducer could be termed a second transducer, and, similarly, a second transducer could be termed a first transducer, without departing from the scope of the various described implementations. The first sensor and the second sensor are both sensors, but they are not the same type of sensor.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. A method of controlling an ultrasound probe, comprising:
at a computing device that includes one or more processors and memory:
during a first portion of a first scan performed by the ultrasound probe, receiving first imaging data acquired via the ultrasound probe, wherein the first imaging data was acquired in accordance with a first set of imaging control parameters, and wherein the first set of imaging control parameters requires that a first subset of a plurality of transducers of the ultrasound probe are activated during the first portion of the first scan; and
during a second portion, after the first portion, of the first scan performed by the ultrasound probe, in accordance with a determination that the first imaging data meets a first set of conditions associated with one or more quality requirements for the first, causing the ultrasound probe to acquire second imaging data in accordance with a second set of imaging control parameters, wherein the second set of imaging control parameters requires that a second subset of the plurality of transducers, different from the first subset of the plurality of transducers, are activated during the second portion of the first scan following the first portion of the first scan.

2. The method of claim 1, further comprising:
in accordance with a determination that the first imaging data does not meet the first set of conditions associated with the one or more quality requirements for the first scan, and in accordance with a determination that the first imaging data meets a second set of conditions associated with the one or more quality requirements for the first scan, causing the ultrasound probe to acquire third imaging data in accordance with a third set of imaging control parameters during the second portion of the first scan following the first portion of the first scan, wherein the third set of imaging control parameters is different from the second set of imaging control parameters.

3. The method of claim 1, further comprising:
in accordance with a determination that the first imaging data does not meet the first set of conditions associated with the one or more quality requirements for the first scan, and in accordance with a determination that the first imaging data does not meet the second set of conditions associated with the one or more quality requirements for the first scan, causing the ultrasound probe to continue using the first set of imaging control parameters to acquire additional imaging data during the second portion of the first scan.

4. The method of claim 1, further comprising:
in accordance with a determination that the first imaging data does not meet the first set of conditions associated with the one or more quality requirements for the first scan:
determining one or more operations for adjusting the ultrasound probe during the second portion of the first scan; and
performing at least one of the one or more operations.

5. The method of claim 4, wherein performing at least one of the one or more operations includes providing guidance for one or more recommended movements to be executed by the ultrasound probe.

6. The method of claim 4, wherein performing at least one of the one or more operations includes adjusting one or more of the first set of imaging control parameters when acquiring a next ultrasound image using the ultrasound probe.

7. The method of claim 1, further comprising:
prior to receiving the first imaging data, determining a respective scan type for the first scan, wherein the respective scan type corresponds to a respective target anatomical structure; and
selecting the first set of image control parameters based at least in part on the respective scan type that is determined for the first scan.

8. The method of claim 7, further comprising:
determining the one or more quality requirements for the first scan according to the respective scan type for the first scan.

9. The method of claim 1, wherein determining whether the first imaging data meets the first set of conditions includes:
determining a respective value of a first quality measure for a first ultrasound image in the first imaging data; and
determining that the first imaging data meets the first set of conditions in accordance with a determination that the respective value of the first quality measure for the first ultrasound image exceeds a first threshold value for the first quality measure.

10. The method of claim 1, wherein determining whether the first imaging data meets the first set of conditions includes:
predicting a respective value for a first quality measure for a next ultrasound image to be acquired in accordance with the first set of imaging control parameters; and
determining that the first imaging data meets the first set of conditions in accordance with a determination that the respective value of the first quality measure for the next ultrasound image to be acquired in accordance with the first set of imaging control parameters would exceed a first threshold value for the first quality measure.

11. The method of claim 10, further comprising:
predicting the respective value of the first quality measure for the next ultrasound image to be acquired based on a predicted trajectory of the ultrasound probe.

12. A computing device, comprising:
one or more processors; and
memory; and
one or more programs stored in the memory and configured for execution by the one or more processors, the one or more programs comprising instructions for:
during a first portion of a first scan performed by the ultrasound probe, receiving first imaging data acquired via the ultrasound probe, wherein the first imaging data was acquired in accordance with a first set of imaging control parameters, and wherein the first set of imaging control parameters requires that a first subset of a plurality of transducers of the ultrasound probe are activated during the first portion of the first scan; and
during a second portion, after the first portion, of the first scan performed by the ultrasound probe, in accordance with a determination that the first imaging data meets a first set of conditions associated with one or more quality requirements for the first, causing the ultrasound probe to acquire second imaging data in accordance with a second set of imaging control parameters, wherein the second set of imaging control parameters requires that a second subset of the plurality of transducers, different from the first subset of the plurality of transducers, are activated during the second portion of the first scan following the first portion of the first scan.

13. The computing device of claim 12, the one or more programs further comprising instructions for:
in accordance with a determination that the first imaging data does not meet the first set of conditions associated with the one or more quality requirements for the first scan, and in accordance with a determination that the first imaging data meets a second set of conditions associated with the one or more quality requirements for the first scan, causing the ultrasound probe to acquire third imaging data in accordance with a third set of imaging control parameters during the second portion of the first scan following the first portion of the first scan, wherein the third set of imaging control parameters is different from the second set of imaging control parameters.

14. The computing device of claim 12, the one or more programs further comprising instructions for:
in accordance with a determination that the first imaging data does not meet the first set of conditions associated with the one or more quality requirements for the first scan, and in accordance with a determination that the first imaging data does not meet the second set of conditions associated with the one or more quality requirements for the first scan, causing the ultrasound probe to continue using the first set of imaging control parameters to acquire additional imaging data during the second portion of the first scan.

15. The computing device of claim 12, the one or more programs further comprising instructions for:
in accordance with a determination that the first imaging data does not meet the first set of conditions associated with the one or more quality requirements for the first scan:
determining one or more operations for adjusting the ultrasound probe during the second portion of the first scan; and
performing at least one of the one or more operations.

16. The computing device of claim 15, wherein the instructions for performing at least one of the one or more operations include instructions for providing guidance for one or more recommended movements to be executed by the ultrasound probe.

17. A non-transitory computer readable storage medium storing one or more programs that, when executed by a computing device having one or more processors and memory, cause the computing device to perform operations comprising:
during a first portion of a first scan performed by the ultrasound probe, receiving first imaging data acquired via the ultrasound probe, wherein the first imaging data was acquired in accordance with a first set of imaging control parameters, and wherein the first set of imaging control parameters requires that a first subset of a plurality of transducers of the ultrasound probe are activated during the first portion of the first scan; and
during a second portion, after the first portion, of the first scan performed by the ultrasound probe, in accordance with a determination that the first imaging data meets a first set of conditions associated with one or more quality requirements for the first, causing the ultrasound probe to acquire second imaging data in accordance with a second set of imaging control parameters, wherein the second set of imaging control parameters requires that a second subset of the plurality of transducers, different from the first subset of the plurality of transducers, are activated during the second portion of the first scan following the first portion of the first scan.

18. The non-transitory computer readable storage medium of claim 17, wherein determining whether the first imaging data meets the first set of conditions includes:
determining a respective value of a first quality measure for a first ultrasound image in the first imaging data; and
determining that the first imaging data meets the first set of conditions in accordance with a determination that the respective value of the first quality measure for the first ultrasound image exceeds a first threshold value for the first quality measure.

19. The non-transitory computer readable storage medium of claim 17, wherein determining whether the first imaging data meets the first set of conditions includes:
predicting a respective value for a first quality measure for a next ultrasound image to be acquired in accordance with the first set of imaging control parameters; and
determining that the first imaging data meets the first set of conditions in accordance with a determination that the respective value of the first quality measure for the next ultrasound image to be acquired in accordance with the first set of imaging control parameters would exceed a first threshold value for the first quality measure.

20. The non-transitory computer readable storage medium of claim 19, the operations further comprising:
predicting the respective value of the first quality measure for the next ultrasound image to be acquired based on a predicted trajectory of the ultrasound probe.
